(19) United States
(12) Patent Application Publication
(10) Pub. No.: US 2023/0293580 A1
(43) Pub. Date: Sep. 21, 2023

(54) SARS-COV2-SPECIFIC T CELL COMPOSITIONS AND THEIR USE IN TREATING AND PREVENTING CORONAVIRUS AND OTHER RESPIRATORY VIRUS INFECTIONS

(71) Applicant: Baylor College of Medicine, Houston, TX (US)

(72) Inventors: Ann Marie Leen, Houston, TX (US); Spyridoula Vasileiou, Houston, TX (US)

(21) Appl. No.: 17/906,490

(22) PCT Filed: Mar. 19, 2021
(86) PCT No.: PCT/US2021/070291

§ 371 Date: Sep. 16, 2022

Related U.S. Application Data
(60) Provisional application No. 63/109,456, filed on Nov. 4, 2020.
(60) Provisional application No. 63/081,441, filed on Sep. 22, 2020.
(60) Provisional application No. 62/992,833, filed on Mar. 20, 2020.

(51) Int. Cl. A61K 35/17; C12N 5/0783; C12N 5/0784; A61P 31/12; A61P 37/04

(52) U.S. Cl. CPC A61K 35/17; A61P 31/12; A61P 37/04; C12N 5/0636; C12N 5/0638; C12N 5/0639; A61K 2039/5158

(57) ABSTRACT

Embodiments of the disclosure concern polyclonal SARS-CoV2 virus specific T cell lines and methods of using the same to treat and prevent viral infections.

### CROSS-REFERENCE TO RELATED APPLICATIONS

This application claims the benefit of priority to U.S. Provisional Application Nos. 62,992,833, filed Mar. 20, 2020; 63/081,441, filed Sep. 22, 2020; and 63/109,456, filed Nov. 4, 2020, the entire contents of each of which are hereby incorporated by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: BAYMP0299_SeqList_ST25.txt, date recorded: Mar. 18, 2021, file size .42 megabytes).

### TECHNICAL FIELD

Embodiments of the disclosure concern at least the fields of cell biology, molecular biology, immunology, and medicine.

### BACKGROUND

Viral infections are a serious cause of morbidity and mortality after allogenic hematopoietic stem cell transplantation (allo-HSCT), which is the treatment of choice for a variety of disorders. Post-transplant, however, graft versus host disease (GVHD), primary disease relapse and viral infections remain major causes of morbidity and mortality. Respiratory tract infections due to community-acquired respiratory viruses including respiratory syncytial virus, Influenza, parainfluenza virus and human metapneumovirus are detected in up to 40% of allogeneic hematopoietic stem cell transplant recipients in whom they cause severe symptoms including pneumonia and bronchiolitis and can be fatal. Other respiratory viruses including adenoviruses (AdV) and coronaviruses strains including SARS-CoV, SARS-CoV-2, MERS-CoV, and also the endemic CoVs that commonly afflict immunocompromised patients also cause severe symptoms, especially in immunocompromised individuals, and the recent SARS-CoV2 pandemic has clearly exposed how ill-prepared we are to treat and prevent infection. Given the lack of effective antivirals and the data from our group demonstrating that adoptively transferred ex vivo-expanded virus-specific T cells can be clinically beneficial for the treatment of both latent (Epstein-Barr virus, cytomegalovirus, BK virus, human herpesvirus 6) and lytic (adenovirus) viruses, we are investigating the potential for extending this immunotherapeutic approach to respiratory viruses. Although available for some viruses, antiviral drugs are not always effective, highlighting the need for novel therapies. One strategy to treat these viral infections is with adoptive T cell transfer, whereby virus-specific T cells (VSTs) are expanded from the peripheral blood of healthy donors ex vivo and then infused to an individual with a viral infection, a stem cell transplant recipient, for example.

In vitro expanded donor-derived and third party virus-specific T cells targeting Adv, EBV, CMV, BK, HHV6 have shown to be safe when adoptively transferred to stem cell transplant patients with viral infections. Virus-specific T cells reconstituted antiviral immunity for Adv, EBV, CMV, BK and HHV6, were effective in clearing disease, and exhibited considerable expansion in vivo. Adoptively transferred in vitro expanded virus-specific T cells have also been shown to be safe and associated with clinical benefit when adoptively transferred to patients.

Embodiments of the present disclosure satisfy a long-felt need in the art by providing therapies for certain viral antigens, and also for restoring T cell immunity by the ex vivo expanded, non-genetically modified, virus-specific T cells to control viral infection and remove symptoms for the period until the transplant patient’s own immune system is restored.

### SUMMARY OF THE EMBODIMENTS

The present disclosure provides virus-specific T-lymphocytes (VSTs) compositions and methods of using the same to treat or prevent viral infections. In some embodiments, the present disclosure provides a composition comprising a polyclonal population of VSTs that recognize one or more coronavirus antigens and/or one or more SARS-CoV2 antigens. In some embodiments, the VSTs are generated by contacting peripheral blood mononuclear cells (PBMCs) with one or more pepmix libraries, each pepmix library containing a plurality of overlapping peptides spanning all or at least a portion of the SARS-CoV2 antigens. In some embodiments, the VSTs are generated by contacting T cells with antigen presenting cells (APCs) such as dendritic cells (DCs) primed with a plurality of pepmix libraries, each pepmix library containing a plurality of overlapping peptides spanning all or at least a portion of the SARS-CoV2 antigens. In some embodiments, the VSTs are generated by contacting T cells with APCs such as DCs nucleofected with at least one DNA plasmid encoding at least one or a portion of one SARS-CoV2 antigen. In some embodiments, the VSTs are cultured ex vivo in the presence of both IL-7 and IL-4. In some embodiments, the VSTs have expanded sufficiently within 9-18 days of culture such that they are ready for administration to a patient.

In some embodiments, the present disclosure provides a composition comprising a polyclonal population of VST that recognize one or more antigens, or a portion thereof, from SARS-CoV2 and one or more additional antigens, or a portion thereof, from one or more additional virus. In some embodiments, the additional virus comprises a different coronavirus serotype/strain. In some embodiments, the additional virus comprises a β-coronavirus (β-CoV). In some embodiments, the additional virus comprises an alpha-coronavirus (α-CoV). In some embodiments, the additional virus is a β-CoV selected from SARS-CoV, MERS-CoV, HCoV-HKU1, and HCoV-OC43. In some embodiments, the additional virus is an α-CoV selected from HCoV-E229 and HCoV-NL63 In some embodiments, the additional virus comprises another respiratory virus. In some embodiments, the respiratory virus is selected from parainfluenza virus (PIV), respiratory syncytial virus (RSV), Influenza, human metapneumovirus (hMPV), adenovirus (AdV), and combinations thereof. In some embodiments, the respiratory virus comprises PIV, RSV, Influenza, hMPV, and AdV. In some embodiments, the VSTs are generated by contacting PBMCs with a plurality of pepmix libraries, each pepmix library containing a plurality of overlapping peptides spanning all or a portion of a SARS-CoV2 antigen or an antigen from the one or more additional viruses. In some embodiments, the VSTs are generated by contacting T cells with APCs such as DCs primed with a plurality of pepmix libraries, each pepmix library containing a plurality of overlapping peptides spanning all or a portion of a viral antigen, wherein at least one of the plurality of pepmix libraries spans a first antigen from SARS-CoV2 and wherein at least one (or a portion of one) additional pepmix library of the plurality of pepmix libraries spans each second antigen. In some embodiments, the VSTs are generated by contacting T cells with APCs such as DCs nucleofected with at least one DNA plasmid encoding at least one SARS-CoV2 antigen, or a portion thereof, and at least one DNA plasmid encoding each second antigen, or a portion thereof. In some embodiments, the plasmid encodes at least one SARS-CoV2 antigen, or a portion thereof, and at least one of the additional antigens, or a portion thereof. In some embodiments, the VSTs comprise CD4+ T-lymphocytes and CD8+ T-lymphocytes. In some embodiments, the VSTs express αβ T cell receptors. In some embodiments, the VSTs comprise effector memory T cells and central memory T cells. In some embodiments, the VSTs are MHC-restricted. In some embodiments, the SARS-CoV2 antigen comprises one or more antigens selected from the group consisting of nsp1; nsp3; nsp4; nsp5; nsp6; nsp10; nsp12; nsp13; nsp14; nsp15; and nsp16. In some embodiments, the SARS-CoV2 antigen comprises one or more antigen selected from the group consisting of Spike (S); Envelope protein (E); Matrix protein (M); and Nucleocapsid protein (N). In some embodiments, the SARS-CoV2 antigen comprises one or more antigen selected from the group consisting of SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14). In some embodiments, the SARS-CoV2 antigens are selected from S, M, N, nsp4, and AP7A, or a combination thereof. In some embodiments, the SARS-CoV2 antigens further comprise nsp3, nsp6, and/or nsp12. In some embodiments, the SARS-CoV2 antigens consist of S, M, N, nsp4, and AP7A. In some embodiments, the present disclosure provides a composition comprising a polyclonal population of VSTs that recognize the SARS-CoV2 antigens S, M, N, nsp4, and AP7A.

In some embodiments, the additional antigen from the additional virus is selected from the group consisting of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, hMPV antigen N, and AdV antigen Hexon, AdV antigen Penton and combinations thereof. In some embodiments, the additional antigen from the additional virus comprises PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, hMPV antigen N, AdV antigen Hexon, AdV antigen Penton and combinations thereof. In some embodiments, the VSTs are cultured ex vivo in the presence of both IL-7 and IL-4. In some embodiments, the VSTs have expanded sufficiently within 9-18 days of culture such that they are ready for administration to a patient. In some embodiments, the VSTs exhibit one or more property selected from (a) negligible alloreactivity; (b) less activation induced cell death of antigen-specific T cells harvested from a patient than corresponding antigen-specific T cells harvested from the same patient, but not cultured in the presence of both IL-7 and IL-4; and (c) viability of greater than 70%. In some embodiments, the composition is negative for bacteria and fungi for at least 7 days in culture; exhibit less than 5 EU/ml of endotoxin, and are negative for mycoplasma. In some embodiments, the pepmixes are chemically synthesized and are >70% pure. In some embodiments, the VSTs are Th1 polarized. In some embodiments, the VSTs produce effector cytokines/molecules including IFN-gamma, TNF-alpha, GM-CSF, Granzyme-B, or perforin upon exposure to antigen. In some embodiments, the VSTs are able to lyse viral antigen-expressing targets cells. In some embodiments, the VSTs do not significantly lyse non-infected autologous or allogenic target cells.

In some embodiments, the present disclosure also provides universal VSTs (UVSTs). In some embodiments, the UVSTs provided herein comprise pooled polyclonal populations of SARS-CoV2-specific VSTs. In some embodiments, the UVSTs provided herein comprise pooled polyclonal populations of pan-coronavirus-specific VSTs.

The terms UVST, universal antigen specific T cell composition, universal cell therapy product, universal antigen-specific cell therapy product, and the like are used interchangeably herein and refer to a cell therapy composition that comprises two or more antigen-specific T cell lines comprising populations of antigen-specific T cells as described herein, wherein said antigen-specific T cell lines are derived from donor material (e.g., MNCs or PBMCs) originating from at least two separate donors. The universal antigen-specific T cell therapy products and/or plurality of antigen-specific T cell lines may be in the form of a composition comprising each antigen-specific T cell line making up the product (i.e., two or more antigen-specific T cell lines), or may be in the form of a plurality of compositions of individual antigen-specific T cell lines for administration in a single dosing session. In embodiments, the universal antigen-specific T cell therapy product comprises a plurality of individual antigen-specific T cell lines generated from a suitable donor population. A suitable donor population may comprise a plurality of different donors, wherein the HLA type of each donor differs from at least one of the other donors on at least one HLA allele, as further described herein. In some embodiments, the universal antigen-specific T cell therapy product (e.g., a UVST) comprises a plurality of cell lines present in a donor bank of cell lines.

In embodiments, the plurality of different donors, the HLA type of each donor differs from at least one of the other donors on at least one HLA allele. In embodiments, the HLA type of each donor differs from at least one of the other donors on at least two HLA alleles. In an aspect, the universal antigen-specific T cell compositions and products include T cells from a sufficient diversity of donors having diversity of HLA alleles such that the compositions and products achieve a high degree of matching across the entire patient population. In various embodiments, the plurality of different donors have sufficient diversity of HLA alleles (with respect to one another) such that the compositions and products match a large percentage of patients across an entire patient population on at least one HLA allele (e.g., 95% or more of a given patient population); for example, in particular aspects such composition and products match 95% or more of patients across an entire patient population on at least two HLA alleles.

In some embodiments, the UVSTs provided herein comprise a combination of SARS-CoV2-specific VSTs with pan-coronavirus-specific VSTs. In some embodiments, the UVSTs provided herein comprise a combination of SARS-CoV2-specific VSTs and/or pan-coronavirus-specific VSTs in combination with VSTs specific for other viruses (e.g., EBV, CMV, Adenovirus, BK, JC virus, HHV6, RSV, Influenza, Parainfluenza, Bocavirus, Coronavirus, Rhinovirus, LCMV, Mumps, Measles, human Metapneumovirus, Parvovirus B, Rotavirus, Merkel cell virus, herpes simplex virus, HPV, HIV, HTLV1, HHV8, West Nile Virus, zika virus, Ebola, and any combination thereof). In some embodiments, the UVSTs provided herein comprise a combination of SARS-CoV2-specific VSTs and/or pan-coronavirus-specific VSTs in combination with VSTs specific for other viruses (e.g., EBV, CMV, Adenovirus, BK, HHV6, and any combination thereof). In some embodiments, the UVSTs provided herein comprise a combination of SARS-CoV2-specific VSTs and/or pan-coronavirus-specific VSTs in combination with VSTs specific for other respiratory viruses (e.g., RSV, influenza, PIV, hMPV, and any combination thereof).

In some embodiments, the present disclosure also provides a pharmaceutical composition comprising any one of the compositions disclosed herein, formulated for intravenous delivery, wherein the composition is negative for bacteria and fungi for at least 7 days in culture; exhibit less than 5 EU/ml of endotoxin, and are negative for mycoplasma.

In some embodiments, the present disclosure provides a method of lysing a target cell comprising contacting the target cell with any one of the compositions disclosed herein (e.g., a composition comprising a plurality of SARS-CoV2 specific VSTs or comprising a multivirus specific population of polyclonal VSTs comprising SARS-CoV2 specific VSTs disclosed herein and VSTs with specificity for any one or more additional virus disclosed herein or a composition comprising a universal antigen-specific T cell therapy product disclosed herein). In some embodiments, the contacting occurs in vivo in a subject. In some embodiments, the contacting occurs in vivo via administration of the VSTs to a subject.

In some embodiments, the present disclosure provides a method of treating or preventing a viral infection comprising administering to a subject in need thereof any one or more of the compositions disclosed herein (e.g., a composition comprising a plurality of SARS-CoV2 specific VSTs or comprising a multivirus specific population of polyclonal VSTs comprising SARS-CoV2 specific VSTs disclosed herein and VSTs with specificity for any one or more additional virus disclosed herein or a composition comprising a universal antigen-specific T cell therapy product disclosed herein). Thus, in embodiments, the present disclosure provides a method of treating or preventing a viral infection comprising administering to a subject in need thereof a composition comprising a polyclonal population of VSTs that recognize the SARS-CoV2 antigens S, M, N, nsp4, and AP7A.

In some embodiments, the method comprises administering between 5x10⁶ and 5x10⁷ VST/m2 to the subject. In some embodiments, the subject is immunocompetent or immunocompromised. In some embodiments, the subject is infected with SARS-CoV2 or has been diagnosed with COVID-19. In some embodiments, the subject has a hematologic malignancy. In some embodiments, the subject has acute myeloid leukemia, acute lymphoblastic leukemia, or chronic granulomatous disease. In some embodiments, the subject, prior to receiving the VSTs, received (a) a matched related donor transplant with reduced intensity conditioning; (b) a matched unrelated donor transplant with myeloablative conditioning; (c) a haplo-identical transplant with reduced intensity conditioning; or (d) a matched related donor transplant with myeloablative conditioning. In some embodiments, the subject (a) has received a solid organ transplantation; (b) has received chemotherapy; (c) has an HIV infection; (d) has a genetic immunodeficiency; and/or (e) has received an allogeneic stem cell transplant; or (f) has received an autologous stem cell transplant. In some embodiments, the subject has cardiovascular disease (including, for example, heart failure, coronary artery disease, and/or cardiomyopathies), diabetes, chronic respiratory disease (for example, COPD), hypertension, cancer, obesity, chronic kidney disease, Down syndrome, immunocompromised state (for example, from stem cell or solid organ transplant), pregnancy, sickle cell disease, and/or is a smoker.

In some embodiments, the composition is administered to the subject a plurality of times. In some embodiments, the administration of the composition effectively treats or prevents a SARS-CoV2 infection in the subject. In some embodiments, the composition effectively treats or prevents a viral infection in the subject, wherein the viral infection is selected from the group consisting of SARS-CoV2, PIV, RSV, Influenza, hMPV, AdV and a combination thereof. In some embodiments, the subject is a human.

In some embodiments, the present disclosure provides a method of treating or preventing a coronavirus infection in a subject comprising administering to the subject a polyclonal population of VSTs generated by a method selected from: (a) contacting PBMCs with one or more pepmix libraries, each pepmix library containing a plurality of overlapping peptides spanning all or at least a portion of one or more SARS-CoV2 antigens; (b) contacting T cells with APCs such as dendritic cells (DCs) primed with a plurality of pepmix libraries, each pepmix library containing a plurality of overlapping peptides spanning all or at least a portion of one or more SARS-CoV2 antigens; or (c) contacting T cells with APCs such as DCs nucleofected with at least one DNA plasmid encoding at least one SARS-CoV2 antigen, or a portion thereof. In some embodiments, the VSTs are cultured ex vivo in the presence of both IL-7 and IL-4. In some embodiments, the VSTs have expanded sufficiently within 9-18 days of culture such that they are ready for administration to a patient. In some embodiments, at least one of the SARS-CoV2 antigens is selected from the group consisting of nsp1; nsp3; nsp4; nsp5; nsp6; nsp10; nsp12; nsp13; nsp14; nsp15; and nsp16. In some embodiments, at least one of the SARS-CoV2 antigens is selected from the group consisting of Spike (S); Envelope protein (E); Matrix protein (M); and Nucleocapsid protein (N). In some embodiments, at least one of the SARS-CoV2 antigens is selected from the group consisting of SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14). In some embodiments, the SARS-CoV2 antigens are selected from the group consisting of nsp1; nsp3; nsp4; nsp5; nsp6; nsp10; nsp12; nsp13; nsp14; nsp15; nsp16; Spike (S); Envelope protein (E); Matrix protein (M); and Nucleocapsid protein (N); SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14), and combinations thereof. In some embodiments, the SARS-CoV2 antigens are selected from the group consisting of S, M, N, nsp3, nsp4, nsp6, nsp12, and AP7A, and combinations thereof. In some embodiments, the SARS-CoV2 antigens are S, M, N, nsp4, and AP7A.

In some embodiments, the coronavirus is a β-coronavirus (β-CoV). In some embodiments, the coronavirus is an alpha-coronavirus (α-CoV). In some embodiments, the β-CoV is SARS-CoV2. In some embodiments, the β-CoV is selected from SARS-CoV, MERS-CoV, HCoV-HKU1, and HCoV-OC43 In some embodiments, the α-CoV is selected from E229 and NL63. In some embodiments, SARs-CoV2 has been detected in the subject (e.g., prior to treatment). In some embodiments, the subject has been diagnosed with COVID-19. In some embodiments, the subject is over 40 years of age. In some embodiments, the subject is over 60 years of age. In some embodiments, the subject is under 40 years of age. In some embodiments, the subject is at a higher risk of an adverse outcome caused by the coronavirus infection due to a preexisting condition In some embodiments, the subject is at a higher risk of dying as a result of the coronavirus infection due to a preexisting condition. In some embodiments, the preexisting condition is selected from cardiovascular disease, diabetes, chronic respiratory disease, hypertension, cancer, obesity, and a combination thereof.

In some embodiments, the present disclosure provides a plurality of compositions, each according to any one of the compositions disclosure herein (e.g., a composition comprising a plurality of SARS-CoV2 specific VSTs or comprising a multivirus specific population of polyclonal VSTs comprising SARS-CoV2 specific VSTs disclosed herein and VSTs with specificity for any one or more additional virus disclosed herein), wherein each composition comprises a polyclonal population of VSTs that differs from one another only in that they were produced from donor PBSTs obtained from different donors. In some embodiments, each of the donors comprise a different HLA type. In some embodiments, the subject is administered the plurality of compositions. In some embodiments, the plurality of compositions are administered to the subject simultaneously. In some embodiments, the plurality of compositions are pooled together prior to administration to the subject. In some embodiments, the pooled composition is cryopreserved and stored as a universal antigen-specific T cell product, which may be thawed prior to administration to the subject. In some embodiments, the VSTs are individually cryopreserved and stored, which may be thawed and pooled prior to administration to the subject or thawed and administered sequentially to the subject. In some embodiments, the plurality of compositions are administered to the subject at different times. In some embodiments, the plurality of compositions comprise enough HLA variability with respect to one another that greater than 95% of the target patient population will be an HLA match with at least one of the plurality of compositions on two or more HLA alleles.

### BRIEF DESCRIPTION OF THE DRAWINGS

FIG. 1 shows β-CoV family members - homology to SARS-CoV2

FIG. 2 shows a schematic of the SARS-CoV2 viral structure

FIG. 3 shows the SARS-CoV2 genomic structure.

FIG. 4 shows exemplary candidate SARS-CoV2 structural and accessary protein antigens

FIG. 5 shows candidate SARS-CoV2 non-structural antigens.

FIG. 6 shows a revised exemplary list of the candidate SARS-CoV2 structural and accessory antigens.

FIG. 7 is a schematic depiction of a process to activate and expand SARS CoV2-specific T cells from immune donors.

FIG. 8 shows the immunodominant antigens in immune donors as measured by the number of IFNγ positive cells and number of responding donors.

FIG. 9 shows the phenotype of the expanded VSTs. The percent of cells in the expanded population expressing the indicated marker is shown.

FIG. 10 is a set of flow cytometry dot plots showing polyfunctionality of the expanded VSTs. The left panel shows IFNγ and TNFα production in CD3+ T cells. The right panel confirms that antiviral activity was detected in both CD4+ and CD8+ T cells as assessed by IFNγ production measured by intracellular cytokine staining (ICS).

FIG. 11 shows polyfunctionality as assessed on the single cell level by CD8+ and CD4+ T cells as measured by isoplexus. Summary polyfunctionality data is shown on the right.

FIG. 12 shows that the expanded VSTs exhibit cytolytic activity against SARS-CoV2-expressing target cells, but not control cells (left panel); and that the expanded VSTs exhibit no activity against non-infected allogeneic or autologous target cells.

FIG. 13 provides a summary of preclinical information regarding SARS-CoV2 specific VSTs.

FIG. 14 provides an exemplary patient population and potential risk factors for the clinical trial described in Example 3.

FIG. 15 shows an exemplary clinical trial design.

FIG. 16A provides the amino acid sequence of the SPIKE protein of SARS2 strain P0DTC2. Regions of the protein that are mutated in the UK variant (B1.1.7 lineage -201/501Y.V1), South African variant (B.1.351 lineage - 20H/501Y.V2), or Brazilian variant (P.1 lineage - 201/501Y.V3) are indicated by the boxed text. Unique immunogenic HLA-restricted epitopes utilized in the study described at Example 5 are indicated with bold, underlined text. FIG. 16B shows the sequences of the indicated regions in wild type and variant strains.

FIG. 17 is a graph showing that the UVSTs did not exhibit auto-reactivity against donor PHA blasts (Donor 1 and Donor 3) and did not exhibit allo-reactivity against unrelated donor PHA blasts (Donor 4 and Donor 5).

### DETAILED DESCRIPTION

### Definitions

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The term “about” when immediately preceding a numerical value means ± 0% to 10% of the numerical value, ± 0% to 10%, ± 0% to 9%, ± 0% to 8%, ± 0% to 7%, ± 0% to 6%, ± 0% to 5%, ± 0% to 4%, ± 0% to 3%, ± 0% to 2%, ± 0% to 1%, ± 0% to less than 1%, or any other value or range of values therein. For example, “about 40” means ± 0% to 10% of 40 (i.e., from 36 to 44).

The use of the term “or” in the claims is used to mean “and/or” unless explicitly indicated to refer to alternatives only or the alternatives are mutually exclusive, although the disclosure supports a definition that refers to only alternatives and “and/or.”

The term “viral antigen” as used herein refers to an antigen that is protein in nature. In specific embodiments, a viral antigen is a coat protein.

Specific examples of viral antigen include at least a virus selected from EBV, CMV, Adenovirus, BK, JC virus, HHV6, RSV, Influenza, Parainfluenza, Bocavirus, Coronavirus, Rhinovirus, LCMV, Mumps, Measles, human Metapneumovirus, Parvovirus B, Rotavirus, Merkel cell virus, herpes simplex virus, HPV, HIV, HTLV1, HHV8, West Nile Virus, zika virus, and Ebola.

The term “antigen-specific T cell lines” or “virus-specific T cells” or “virus-specific T cell lines” are used interchangeably herein to refer to polyclonal T cell lines that have specificity and potency against a virus or viruses of interest. As described herein, a viral antigen or several viral antigens are presented to native T cells in peripheral blood mononuclear cells and the native CD4 and CD8 T cell populations expand in response to said viral antigen(s). For example, an antigen-specific T cell line or a virus-specific T cell for EBV can recognize EBV, thereby expanding the T cells specific for EBV. In another example, an antigen-specific T cell line or a virus-specific T cell for adenovirus and BK can recognize both adenovirus and BK, thereby expanding the T cells specific for adenovirus and BK.

The terms universal antigen specific T cell composition, universal cell therapy product, universal antigen-specific cell therapy product, universal virus-specific T cell product, UVST, and the like are used interchangeably herein and refer to a cell therapy composition that comprises two or more virus-specific T cell lines comprising populations of virus-specific T cells as described herein, wherein said virus-specific T cell lines are derived from donor material (e.g., MNCs or PBMCs) originating from a plurality of different donors (i.e., at least two separate donors). Universal VSTs have been described, for example, in PCT/US2021/016266, which is hereby incorporated by reference in its entirety. In some embodiments, the HLA type of each donor in the plurality of different donors differs from at least one of the other donors in the plurality of different donors on at least one HLA allele. In an aspect, the UVST compositions and products include VST cell lines from a sufficient diversity of donors having diversity of HLA alleles such that the compositions and products achieve a high degree of matching across the entire patient population. In various embodiments, the plurality of different donors have sufficient diversity of HLA alleles (with respect to one another) such that the compositions and products match a large percentage of patients across an entire patient population on at least one HLA allele (e.g., 95% or more of a given patient population); for example, in particular aspects such composition and products match 95% or more of patients across an entire patient population on at least two HLA alleles. The patient population may be any target population, and the selection of the donors may occur with actual knowledge of the HLA type of that patient population or with knowledge of the average HLA types of that population. For example, in some aspects, the population may be the entire US population or the entire world population. In some aspects, the population may be the entire US allogeneic HSCT population. In aspects the population may be the entire world allogeneic HSCT population.

As used herein, the terms “patient” or “subject” are used interchangeably herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats and horses. Examples of agricultural animals include pigs, cattle and goats.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. In some instances, treatment is curative or ameliorating.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

As used herein, the terms “comprise,” “comprising,” “includes,” “including,” “has,” “having,” “contains,” “containing,” “characterized by,” or any other variation thereof, are intended to encompass a non-exclusive inclusion, subject to any limitation explicitly indicated otherwise, of the recited components. For example, a composition and/or method that “comprises” a list of elements (e.g., components or features or steps) is not necessarily limited to only those elements (or components or features or steps), but may include other elements (or components or features or steps) not expressly listed or inherent to the composition and/or method.

As used herein, the phrases “consists of” and “consisting of” exclude any element, step, or component not specified. For example, “consist of” or “consisting of” used in a claim would limit the claim to the components, materials or steps specifically recited in the claim except for impurities ordinarily associated with therewith (i.e., impurities within a given component). When the phrase “consist of” or “consisting of” appears in a clause of the body of a claim, rather than immediately following the preamble, the phrase “consist of” or “consisting of” limits only the elements (or components or steps) set forth in that clause; other elements (or components) are not excluded from the claim as a whole.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### Overview

Respiratory viral infections due to community-acquired respiratory viruses (CARVs) including respiratory syncytial virus (RSV), influenza, parainfluenza virus (PIV) and human metapneumovirus (hMPV) are detected in up to 40% of allogeneic hematopoietic stem cell transplant (allo-HSCT) recipients, in whom they may cause severe disease such as bronchiolitis and pneumonia that can be fatal. RSV induced bronchiolitis is the most common reason for hospital admission in children less than 1 year, while the Center for Disease Control (CDC) estimates that, annually, Influenza accounts for up to 35.6 million illnesses worldwide, between 140,000 and 710,000 hospitalizations, annual costs of approximately $87.1 billion in disease management in the US alone and between 12,000 and 56,000 deaths.

Other respiratory viruses including adenovirus (AdV) and the coronavirus strains SARS-CoV, SARS-CoV-2, MERS-CoV, and also the endemic CoVs that afflict both immunocompetent and immunocompromised patients also cause severe symptoms, especially in immunocompromised individuals, and the recent SARS-CoV2 pandemic has clearly exposed how ill-prepared we are to treat and prevent infection. This horrible pandemic has already resulted in millions of deaths worldwide, the collapse of healthcare systems, and a global economic meltdown not seen in decades. Thus, it is clear there is an urgent need for new therapies to treat these viruses.

The present disclosure provides restoration of T cell immunity by the administration of ex vivo expanded, non-genetically modified, virus-specific T cells (VSTs) to control viral infections and eliminate symptoms. Without wishing to be bound by any theories, VSTs recognize and kill virus-infected cells via their native T cell receptor (TCR), which binds to major histocompatibility complex (MHC) molecules expressed on target cells that present virus-derived peptides. Thus, in some embodiments, the SARS-CoV2 specific VSTs disclosed herein are useful for treating or preventing SARS-CoV2 infections, and/or treating or preventing other coronavirus infections.

In some embodiments, VSTs are produced from mononuclear cells (MNCs) procured from healthy, pre-screened, seropositive donors, which are available as a partially HLA-matched “off-the-shelf” product. In some embodiments, VSTs are produced from peripheral blood mononuclear cells (PBMCs) procured from healthy, pre-screened, seropositive donors, which are available as a partially HLA-matched “off-the-shelf” product. In some embodiments, the donor may be a person who has recovered from a SARS-CoV2 infection with either the parental or a variant strain. In some embodiments, the VSTs as described herein respond to (or “are specific for”) at least SARS-CoV2. In some embodiments, the VSTs as described herein are multivirus specific T cells that respond to SARS-CoV2 and one or more additional viruses. In some embodiments, the VSTs as described herein respond to SARS-CoV2 and one or more additional coronavirus. In some embodiments, the additional coronavirus is an alpha coronavirus. In particular embodiments, the alpha coronavirus is selected from HCoV-E229 and HCoV-NL63. In some embodiments, the additional coronavirus is a beta coronavirus. In some embodiments, the beta coronavirus is selected from SARS-CoV, MERS-CoV, HCoV-HKU1, and HCoV-OC43. In some embodiments, the VSTs described herein respond to SARS-CoV2 and one or more additional respiratory virus. In some embodiments the additional respiratory virus is selected from parainfluenza virus (PIV), respiratory syncytial virus (RSV), Influenza, human metapneumovirus (hMPV), adenovirus (AdV), and combinations thereof. In one particular embodiment, the VSTs are specific for SARS-CoV2, PIV, RSV, Influenza, hMPV, AdV. In some embodiments, the VSTs described herein respond to parental and SARS-CoV2 variants. For example, in some embodiments, the VSTs described herein respond to the parental and one or more of the D614G variant of SARS-CoV2, one or more UK variant (e.g., B.1.1.7 lineage -201/501Y.V1), one or more UK/South African variant (e.g., B.1.351 lineage - 20H/501Y.V2), one or more Brazilian variant (P.1 lineage - 20J/501Y.V3), and/or one or more additional variant known, later discovered, and/or later emerging.

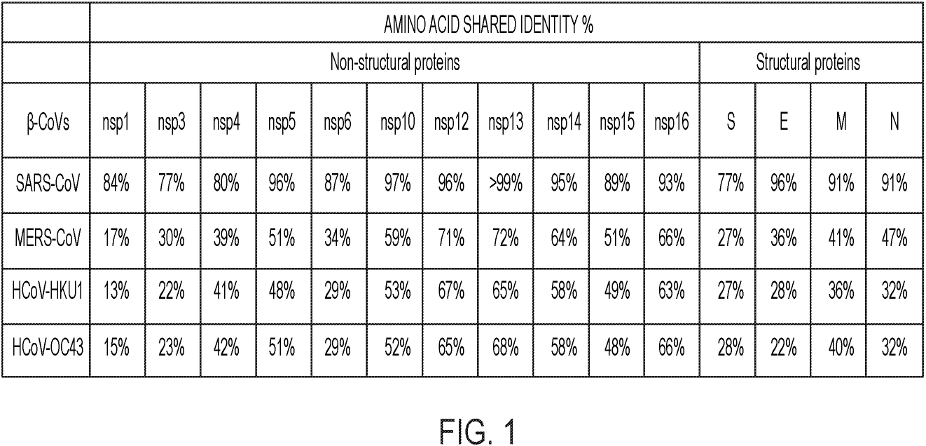

SARS-CoV2 is a beta-coronavirus (β-CoV) related to the SARS coronavirus SARS-CoV. As shown in FIG. 1, SARS-CoV2 shares a high degree of homology with SARS-CoV and to a lesser extent with other members of the β-CoV family:

SARS-CoV2 is the virus responsible for COVID-19, which was first seen in Wuhan, China in 2019 and was declared a global pandemic in early 2020. COVID-19 commonly causes symptoms including fever, cough, and shortness of breath, and may in some rarer cases cause muscle pain, sputum production and sore throat. In rare instances, the disease progresses to severe pneumonia and multi-organ failure. In particular, although the disease is still in its infancy and predispositions to adverse outcomes are still changing, it appears that the severe progression is more common in older individuals and in individuals with comorbidities such as (but not limited to) cardiovascular disease (including, for example, heart failure, coronary artery disease, and/or cardiomyopathies), diabetes, chronic respiratory disease (for example, COPD), hypertension, cancer, obesity, chronic kidney disease, Down syndrome, immunocompromised state (for example, from stem cell or solid organ transplant), pregnancy, sickle cell disease, smoking, and any combination thereof. In some embodiments, the present disclosure provides methods for treating a patient having one or more of these comorbidities, and/or COVID-19 comorbidities identified in the future, by administering the VSTs provided herein. Thus, in some embodiments, the present disclosure provides methods for treating or preventing a SARS-CoV2 infection and/or COVID-19 in a patient in need thereof, wherein the patient has one or more of a cardiovascular disease (e.g., heart failure, coronary artery disease, and/or cardiomyopathies), diabetes, chronic respiratory disease (e.g., COPD), hypertension, cancer, obesity, chronic kidney disease, Down syndrome, immunocompromised state (e.g., from stem cell or solid organ transplant), pregnancy, and sickle cell disease.

In some embodiments, the VSTs disclosed herein that are specific for SARS-CoV2 also display cross reactivity against other coronaviruses. Thus, while the SARS-CoV2 specific VSTs disclosed herein may in some embodiments be utilized for treating or preventing SARS-CoV2 infections, they may additionally or alternatively be utilized for treating other coronavirus infections in some embodiments. For example, in one embodiment the SARS-CoV2 specific VSTs disclosed herein are used to treat or prevent a SARS-CoV infection. In one embodiment the SARS-CoV2 specific VSTs disclosed herein are used to treat or prevent a MERS-CoV infection. In one embodiment the SARS-CoV2 specific VSTs disclosed herein are used to treat or prevent an HCoV-HKU1 infection. In one embodiment the SARS-CoV2 specific VSTs disclosed herein are used to treat or prevent an HCoV-OC43 infection. In one embodiment the SARS-CoV2 specific VSTs disclosed herein are used to treat or prevent an alpha coronavirus infection. In one embodiment the SARS-CoV2 specific VSTs disclosed herein are used to treat or prevent an HCoV-E229 infection. In one embodiment the SARS-CoV2 specific VSTs disclosed herein are used to treat or prevent an HCoV-NL63 infection.

### Generation of Pepmix Libraries

In some embodiments of the invention, a library of peptides is provided to PBMCs ultimately to generate VSTs. The library in particular cases comprises a mixture of peptides (“pepmixes”) that span part or all of the same antigen. Pepmixes utilized in the invention may be from commercially available peptide libraries made up of peptides that are 15 amino acids long and overlapping one another by 11 amino acids, in certain aspects. In some cases, they may be generated synthetically. Examples include those from JPT Technologies (Springfield, VA) or Miltenyi Biotec (Auburn, CA). In particular embodiments, the peptides are at least 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example.

In some embodiments, the amino acids as used in the pepmixes have at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least 99.9% purity, inclusive of all ranges and subranges therebetween. In some embodiments, the amino acids as used here in the pepmixes have at least 70% purity.

The mixture of different peptides may include any ratio of the different peptides, although in some embodiments each particular peptide is present at substantially the same numbers in the mixture as another particular peptide. The methods of preparing and producing pepmixes for multiviral cytotoxic T cells with broad specificity is described in US2018/0187152, which is incorporated by reference in its entirety.

### Production of VSTs

In some embodiments, methods of producing VSTs comprise isolating mononuclear cells (MNCs), or having MNCs, isolated, from blood obtained from donors. In some embodiments, the MNCs are PBMCs. MNCs and PBMCs are isolated by using the methods known by a skilled person in the art. By way of examples, density centrifugation (gradient) (Ficoll-Paque) can be used for isolating PBMCs. In other example, cell preparation tubes (CPTs) and SepMate tubes with freshly collected blood can be used for isolating PBMCs.

In some embodiments, the MNCs are PBMCs. By way of example, PBMC can comprise lymphocytes, monocytes, and dendritic cells. By way of example, lymphocytes can include T cells, B cells, and NK cells. In some embodiments, the MNCs as used herein are cultured or cryopreserved. In some embodiments, the process of culturing or cryopreserving the cells can include contacting the cells in culture with one or more antigens under suitable culture conditions to stimulate and expand antigen-specific T cells. In some embodiments, the one or more antigen can comprise one or more viral antigen.

In some embodiments, the process of culturing or cryopreserving the cells can include contacting the cells in culture with one or more epitope from one or more antigen under suitable culture conditions. In some embodiments, contacting the MNCs or PBMCs with one or more antigen, or one or more epitope from one or more antigen, stimulate and expand a polyclonal population of antigen-specific T cells from each of the respective donor’s MNCs or PMBCs. In some embodiments, the antigen-specific T cell lines can be cryopreserved.

In some embodiments, the one or more antigen can be in the form of a whole protein. In some embodiments, the one or more antigen can be a pepmix comprising a series of overlapping peptides spanning part of or the entire sequence of each antigen. In some embodiments, the one or more antigen can be a combination of a whole protein and a pepmix comprising a series of overlapping peptides spanning part of or the entire sequence of each antigen.

In some embodiments, the culturing of the PBMCs or MNCs is in a vessel comprising a gas permeable culture surface. In one embodiment, the vessel is an infusion bag with a gas permeable portion or a rigid vessel. In one embodiment, the vessel is a GRex bioreactor. In one embodiment, the vessel can be any container, bioreactor, or the like, that are suitable for culturing the PBMCs or MNCs as described herein.

In some embodiments, the PBMCs or MNCs are cultured in the presence of one or more cytokine. In some embodiments, the cytokine is IL4. In some embodiments, the cytokine is IL7. In some embodiments, the cytokine is IL4 and IL7. In some embodiments, the cytokine includes IL4 and IL7, but not IL2. In some embodiments, the cytokine can be any combinations of cytokines that are suitable for culturing the PBMCs or MNCs as described herein.

In some embodiments, culturing the MNCs or PBMCs can be in the presence of at least 2, 3, 4, 5, 6, 7, 8, 9, 10, 11, 12, 13, 14, 15, 16, 17, 18, 19, 20, or more different pepmixes. Pepmixes, a plurality of peptides, comprise a series of overlapping peptides spanning part of or the entire sequence of an antigen. In some embodiments, the MNCs or PBMCs can be cultured in the presence of a plurality of pepmixes. In this instance, each pepmix covers at least one antigen that is different than the antigen covered by each of the other pepmixes in the plurality of pepmixes. In some embodiments, at least 2, 3, 4, 5, 6, 7, 8, 9, 10, 11, 12, 13, 14, 15, 16, 17, 18, 19, 20 or more different antigens are covered by the plurality of pepmixes. In some embodiments, at least one antigen from at least 2 different viruses are covered by the plurality of pepmixes.

In some embodiments, the pepmix comprises 15 mer peptides. In some embodiments, the pepmix comprises peptides that are suitable for the methods as described herein. In some embodiments, the peptides in the pepmix that span the antigen overlap in sequence by 8 amino acids, 9 amino acids, 10 amino acids, 11 amino acids, 12 amino acids, 13 amino acids, 14 amino acids, 15 amino acids. In some embodiments, the peptides in the pepmix that span the antigen overlap in sequence by 11 amino acids.

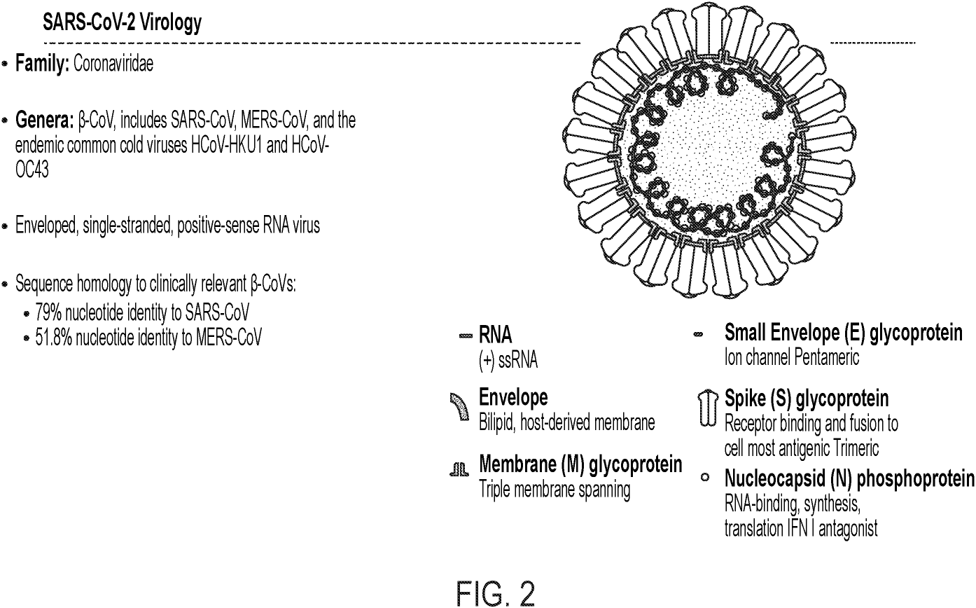

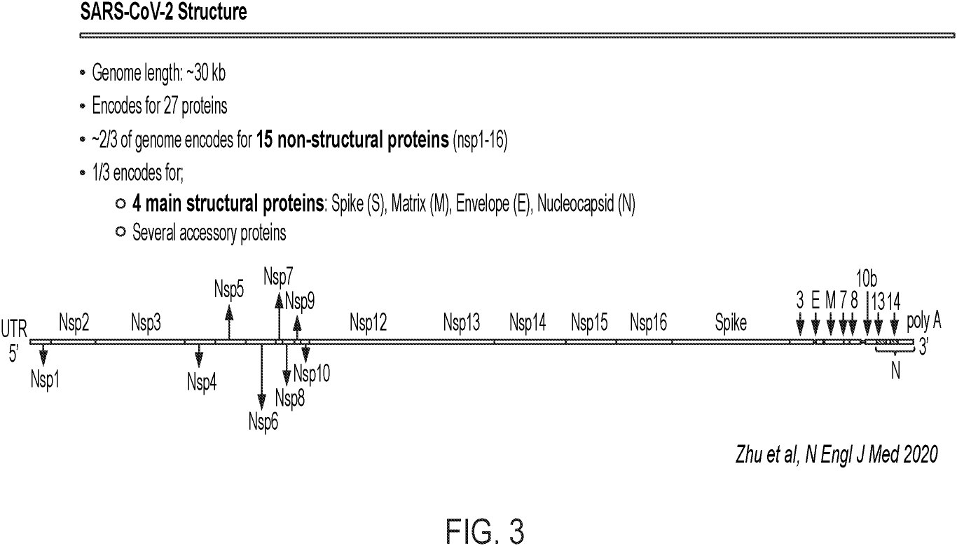

In some embodiments, the viral antigen in the one or more pepmixes is from SARS-CoV2. FIG. 2 shows a schematic of the SARS-CoV2 viral structure and FIG. 3 shows the SARS-CoV2 genomic structure. This virus is an enveloped, single-stranded, positive-sense RNA virus. The SARS-CoV2 genome encodes 27 proteins including 4 main structural proteins: Spike (S), Matrix (M), Envelope (E), Nucleocapsid (N) and several non-structural accessory proteins. The term “Matrix (M) protein” and the like (e.g., M protein or M antigen) is used interchangeably herein with the term “Membrane (M) protein” and the like (e.g., Membrane (M) matrix protein).

In some embodiments, the SARS-CoV2 antigen in the one or more pepmixes comprises one or more structural antigen. In some embodiments, the SARS-CoV2 antigen in the one or more pepmixes comprises one or more non-structural antigen. In some particular embodiments, the antigen in the one or more pepmixes comprises a structural antigen and the antigen in one or more other pepmixes comprises a non-structural antigen.

In some embodiments, the SARS-CoV2 antigen in the one or more pepmixes comprises one or more antigen selected from the group consisting of nsp1; nsp3; nsp4; nsp5; nsp6; nsp10; nsp12; nsp13; nsp14; nsp15; and nsp16. In some embodiments, the SARS-CoV2 antigen comprises one or more antigen selected from the group consisting of Spike (S); Envelope protein (E); Matrix protein (M); and Nucleocapsid protein (N). In some embodiments, the SARS-CoV2 antigen comprises one or more antigen selected from the group consisting of SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14).

In some embodiments, the SARS-CoV2 antigen in the one or more pepmixes comprises one or more antigen selected from the group consisting of nsp1; nsp3; nsp4; nsp5; nsp6; nsp10; nsp12; nsp13; nsp14; nsp15; nsp16; Spike (S); Envelope protein (E); Matrix protein (M); Nucleocapsid protein (N). In some embodiments, the SARS-CoV2 antigen further comprises one or more antigen selected from the group consisting of SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14). In embodiments, the SARS-CoV2 antigens comprise S, M, N, nsp4, AP7A, or any combination thereof. In embodiments, the SARS-CoV2 antigens consist of S, M, N, nsp4, and AP7A.

In embodiments, the present disclosure provides VSTs (e.g., VST compositions) comprising a plurality of VSTs directed to one or more of nsp1; nsp3; nsp4; nsp5; nsp6; nsp7; nsp8; nsp10; nsp12; nsp13; nsp14; nsp15; nsp16; Spike (S); Envelope protein (E); Matrix protein (M); Nucleocapsid protein (N); SARS-CoV-2 (AP3A); SARS-CoV-2 (ORF10); SARS-CoV-2 (ORF9B); SARS-CoV-2 (Y14) SARS-CoV-2 (AP7A); SARS-CoV-2 (AP7B). In some embodiments, the present disclosure provides VSTs (e.g., VST compositions) comprising a plurality of VSTs directed to (i) one or more structural antigen selected from one or more of Spike (S); Envelope protein (E); Matrix protein (M); and Nucleocapsid protein; (ii) one or more nonstructural antigen selected from nsp1; nsp3; nsp4; nsp5; nsp6; nsp7; nsp8 nsp10; nsp12; nsp13; nsp14; nsp15; nsp16; and (iii) one or more antigen selected from SARS-CoV-2 (AP3A); SARS-CoV-2 (ORF10); SARS-CoV-2 (ORF9B); SARS-CoV-2 (Y14); SARS-CoV-2 (AP7A); and SARS-CoV-2 (AP7B). The present disclosure also comprises pharmaceutical compositions comprising such VST compositions.

In embodiments, the present disclosure provides VSTs (e.g., VST compositions) comprising a plurality of VSTs directed to one or more of S, M, N, nsp3, nsp4, nsp6, nsp12, and AP7A. In embodiments, the present disclosure provides VSTs (e.g., VST compositions) consisting of a plurality of VSTs directed to one or more of S, M, N, nsp3, nsp4, nsp6, nsp12, and AP7A. In embodiments, the present disclosure provides VSTs (e.g., VST compositions) consisting essentially of a plurality of VSTs directed to one or more of S, M, N, nsp3, nsp4, nsp6, nsp12, and AP7A. In embodiments, the present disclosure provides VSTs generated by culturing PBMCs in the presence of SARS-CoV2 antigens S, M, N, nsp3, nsp4, nsp6, nsp12, and AP7A. In embodiments, the present disclosure provides VSTs (e.g., VST compositions) comprising a plurality of VSTs directed to the antigens S, M, N, nsp4, and AP7A. In embodiments, the present disclosure provides VSTs (e.g., VST compositions) consisting of a plurality of VSTs directed to the antigens S, M, N, nsp4, and AP7A. In embodiments, the present disclosure provides VSTs (e.g., VST compositions) consisting essentially of a plurality of VSTs directed to the antigens S, M, N, nsp4, and AP7A. In embodiments, the present disclosure provides VSTs generated by culturing PBMCs in the presence of SARS-CoV2 antigens S, M, N, nsp4, and AP7A.

The present disclosure also provides multi-VSTs comprising a plurality of VSTs directed to one or more of the above-mentioned SARS-CoV2 antigens and directed to one or more additional viral antigen. The additional viral antigen may comprise one or more or all of the group consisting of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, hMPV antigen N, and AdV antigen Hexon, AdV antigen Penton and combinations thereof.

The additional viral antigen may also in some embodiments comprise a virus selected from EBV, CMV, Adenovirus, BK, JC virus, HHV6, RSV, Influenza, Parainfluenza, Bocavirus, Rhinovirus, Coronavirus, LCMV, Mumps, Measles, human Metapneumovirus, Parvovirus B, Rotavirus, merkel cell virus, herpes simplex virus, HPV, HIV, HTLV1, HHV8, West Nile Virus, zika virus, and ebola. In some embodiments, at least one pepmix covers an antigen (or part of an antigen) from RSV, Influenza, PIV, HMPV. In some embodiments, the virus can be any suitable viruses.

In some embodiments, the influenza antigens can be influenza A antigen NP1. In some embodiment, the influenza antigens can be influenza A antigens MP1. In some embodiment, the influenza antigens can be a combination of NP1 and MP1. In some embodiments, the RSV antigens can be RSV N. In some embodiments, the RSV antigens can be RSV F. In some embodiments, the RSV antigens can be a combination of RSV N and F. In some embodiments, the hMPV antigens can be F. In some embodiments, the hMPV antigens can be N. In some embodiments, the hMPV antigens can be M2-1. In some embodiments, the hMPV antigens can be M. In some embodiments, the hMPV antigens can be a combination of F, N, M2-1, and M. In some embodiments, the PIV antigens can be M. In some embodiments, the PIV antigens can be HN. In some embodiments, the PIV antigens can be N. In some embodiments, the PIV antigens can be F. In some embodiments, the PIV antigens can be a combination of M, HN, N, and F.

In other embodiments, at least one pepmix covers an antigen from EBV, CMV, adenovirus, BK, and HHV6. In some embodiments, the EBV antigens are from LMP2, EBNA1, BZLF1, and a combination thereof. In some embodiments, the CMV antigens are from IE1, pp65, and a combination thereof. In some embodiments, the adenovirus antigens are from Hexon, Penton, and a combination thereof. In some embodiments, the BK virus antigens are from VP1, large T, and a combination thereof. In some embodiments, the HHV6 antigens are from U90, U11, U14, and a combination thereof.

In some embodiments, the PBMCs or MNCs are cultured in the presence of pepmixes spanning influenza A antigen NP1 and Influenza A antigen MP1, RSV antigens N and F, hMPV antigens F, N, M2-1, and M, and PIV antigens M, HN, N, and F. In some embodiments, the PBMCs or MNCs are cultured in the presence of pepmixes spanning EBV antigens LMP2, EBNA1, and BZLF1, CMV antigens IE1 and pp65, adenovirus antigens Hexon and Penton, BK virus antigens VP1 and large T, and HHV6 antigens U90, U11, and U14. In some embodiments, the antigen specific T cells are tested for antigen-specific cytotoxicity.

The present disclosure provides methods of treating a disease or condition comprising administering to a patient one or more suitable VST cell lines, either sequentially or simultaneously, described herein.

In some embodiments, the patient is infected with SARS-CoV2. In some embodiments, the patient has been diagnosed with COVID-19. In some embodiments, the patient is immunocompromised. As used herein, immunocompromised means having a weakened immune system. For example, patients who are immunocompromised have a reduced ability to fight infections and other diseases. In some embodiments, the patient is immunocompromised due to a treatment the patient received to treat the disease or condition or another disease or condition. In some embodiments, the cause of immunocompromised is due to age. In one embodiment, the cause of immunocompromised is due to young age. In one embodiment, the cause of immunocompromised is due to old age. In some embodiments, the patient is in need of a transplant therapy. In embodiments, the patient has received an organ or tissue transplant. In embodiments, the patient has cancer, e.g., a hematologic malignancy.

In some embodiments, the treatment efficacy is measured post-administration of the VST cell line. In other embodiments, the treatment efficacy is measured based on viremic resolution of infection. In other embodiments, the treatment efficacy is measured based on viruric resolution of infection. In other embodiments, the treatment efficacy is measured based on resolution of viral load in a sample from the patient. In some embodiments, the sample is from a nasal swab. In other embodiments, the treatment efficacy is measured based on viremic resolution of infection, viruric resolution of infection, and resolution of viral load in a sample from the patient. In some embodiments, the treatment efficacy is measured by monitoring viral load detectable in the peripheral blood of the patient. In some embodiments, the treatment efficacy comprises resolution of macroscopic hematuria. In some embodiments, the treatment efficacy comprises reduction of hemorrhagic cystitis symptoms as measured by the CTCAE-PRO or similar assessment tool that examines patient and/or clinician-reported outcomes.

The sample is selected from a tissue sample from the patient. The sample is selected from a fluid sample from the patient. The sample is selected from cerebral spinal fluid (CSF) from the patient. The sample is selected from BAL from the patient. The sample is selected from stool from the patient.

In some embodiments, the one or more second virus can be a PIV antigen. In some embodiments, the PIV antigen can be PIV antigen M. In some embodiments, the PIV antigen can be PIV antigen HN. In some embodiments, the PIV antigen can be PIV antigen N. In some embodiments, the PIV antigen can be PIV antigen F. In some embodiments, the PIV antigen can be any combinations of PIV antigen M, PIV antigen HN, PIV antigen N, and PIV antigen F

In some embodiments, the one or more second virus can be RSV. In some embodiments, the one or more second virus can be Influenza. In some embodiments, the one or more second virus can be hMPV. In some embodiments, the one or more second virus can comprises RSV, Influenza, and human metapneumovirus. In some embodiments, the one or more second virus can consist of RSV, Influenza, and human metapneumovirus. In some embodiments, the one or more second virus can be selected from any suitable viruses as described herein.

In some embodiments, the composition can comprise two or three second viruses. In some embodiments, the composition can comprise three second viruses. In some embodiments, the three second viruses can comprise influenza, RSV, and hMPV. In some embodiments, the composition comprise at least two second antigens per each second virus. In some embodiments, the composition comprises 1 second antigen. In some embodiments, the composition comprises 2 second antigens. In some embodiments, the composition comprises 3 second antigens. In some embodiments, the composition comprises 4 second antigens. In some embodiments, the composition comprises 5 second antigens. In some embodiments, the composition comprises 6 second antigens. In some embodiments, the composition comprises 7 second antigens. In some embodiments, the composition comprises 8 second antigens. In some embodiments, the composition comprises 9 second antigens. In some embodiments, the composition comprises 10 second antigens. In some embodiments, the composition comprises 11 second antigens. In some embodiments, the composition comprises 12 second antigens. In some embodiments, the composition comprises any numbers of second antigens that would be suitable for the compositions as described herein.

In some embodiments, the second antigen can be influenza antigen NP1. In some embodiments, the second antigen can be influenza antigen MP1. In some embodiments, the second antigen can be RSV antigen N. In some embodiments, the second antigen can be RSV antigen F. In some embodiments, the second antigen can be hMPV antigen M. In some embodiments, the second antigen can be hMPV antigen M2-1. In some embodiments, the second antigen can be hMPV antigen F. In some embodiments, the second antigen can be hMPV antigen N. In some embodiments, the second antigen can be any combinations of influenza antigen NP1, influenza antigen MP1, RSV antigen N, RSV antigen F, hMPV antigen M, hMPV antigen M2-1, hMPV antigen F, and hMPV antigen N.

In some embodiments, the second antigen comprises influenza antigen NP1. In some embodiments, the second antigen comprises influenza antigen MP1. In some embodiments, In some embodiments, the second antigen comprises both influenza antigen NP1 and influenza antigen MP1. In some embodiments, the second antigen comprises RSV antigen N. In some embodiments, the second antigen comprises RSV antigen F. In some embodiments, the second antigen comprises both RSV antigen N RSV antigen F.

In some embodiments, the second antigen comprises hMPV antigen M. In some embodiments, the second antigen comprises hMPV antigen M2-1. In some embodiments, the second antigen comprises hMPV antigen F. In some embodiments, the second antigen comprises hMPV antigen N. In some embodiments, the second antigen comprises combinations of hMPV antigen M, hMPV antigen M2-1, hMPV antigen F, and hMPV antigen N.

In some embodiments, the second antigen comprises each of influenza antigen NP1, influenza antigen MP1, RSV antigen N, RSV antigen F, hMPV antigen M, hMPV antigen M2-1, hMPV antigen F, hMPV antigen N. In some embodiments, the plurality of antigens comprise PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist of PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist essentially of PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the second antigen can comprise any suitable antigens for the compositions as described herein.

In some embodiments, the VSTs in the compositions can be generated by contacting PBMCs with a plurality of pepmix libraries. In some embodiments, each pepmix library contains a plurality of overlapping peptides spanning at least a portion of a viral antigen. In some embodiments, at least one of the plurality of pepmix libraries spans a first antigen from PIV-3. In some embodiments, at least one additional pepmix library of the plurality of pepmix libraries spans each second antigen.

In some embodiments, the VSTs can be generated by contacting T cells with APCs such as dendritic cells (DCs) nucleofected with at least one DNA plasmid. In some embodiments, the DNA plasmid can encode the PIV antigen. In some embodiments, the at least one DNA plasmid encodes each second antigen. In some embodiments, the plasmid encodes at least one PIV antigen and at least one of the second antigens. In some embodiments, the compositions as described herein comprise CD4+ T-lymphocytes and CD8+ T- lymphocytes. In some embodiments, the compositions comprise VSTs expressing αβT cell receptors. In some embodiments, the compositions comprise MHC-restricted CTLs.

In some embodiments, the VSTs can be cultured ex vivo in the presence of both IL-7 and IL-4. In some embodiments, the multivirus VSTs have expanded sufficiently within 9 days, 10 days, 11 days, 12 days, 13 days, 14 days, 15 days, 16 days, 17 days, 18 days, 19 days, 20 days inclusive of all ranges and subranges therebetween, of culture such that they are ready for administration to a patient. In some embodiments, the multivirus VSTs have expanded sufficiently within any number of days that are suitable for the compositions ad described herein.

The present disclosure provides compositions comprising VSTs that exhibit negligible alloreactivity. In some embodiments, the compositions comprising VSTs that exhibit less activation induced cell death of antigen-specific T cells harvested from a patient than corresponding antigen-specific T cells harvested from the same patient. In some embodiments, the compositions are not cultured in the presence of both IL-7 and IL-4. In some embodiments, the compositions comprising CTL exhibit viability of greater than 70% (e.g., viability of at least about 70%, at least about 75%, at least about 80%, at least about 85%, at least about 90%, or at least about 95%).

In some embodiments, the compositions are negative for bacteria and fungi for at least 1 days, at least 2 days, at least 3 days, at least 4 days, at least 5 days, at least 6 days at least 7 days, at least 8 days, at least 9 days, at least 10 days, in culture. In some embodiments, the composition is negative for bacteria and fungi for at least 7 days in culture. In some embodiments, the compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the compositions exhibit less than 5 EU/ml of endotoxin. In some embodiments, the compositions are negative for mycoplasma.

In some embodiments, the pepmixes used for constructing the polyclonal of CTLs are chemically synthesized. In some embodiments, the pepmixes are optionally >10%, >20%, >30%, >40%, >50%, >60%, >70%, >80%, >90%, inclusive of all ranges and subranges therebetween, pure. In some embodiments, the pepmixes are optionally >90% pure.

In some embodiments, at least about 90%, at least about 95%, at least about 96%, at least about 97%, at least about 98%, or at least about 99% of the cells obtained by the methods provided herein are CD3+ T cells. In embodiments, the VSTs comprise both CD4+ and CD8+ T cells. In embodiments, the VSTs include T cells that express the activation markers CD25, CD69, and/or CD28. In embodiments, at least about 20%, at least about 30%, at least about 40%, at least about 50%, at least about 60%, at least about 70%, or at least about 80% of the cells are CD28+ T cells (CD3+ CD28+) and/or CD69+ T cells (CD3+CD69+) and/or CD25+ T cells (CD3+CD25+). In embodiments, the VSTs do not comprise a significant number of exhausted, anergic, or regulatory T cells. For example, in embodiments, the VSTs comprise less than about 10%, less than about 5%, less than about 2%, or less than about 1% PD1+ T cells (CD3+PD1+), and/or comprise less than about 10%, less than about 5%, less than about 2%, or less than about 1% TIM3+ T cells (CD3+TIM3+). In some embodiments, the VSTs are substantially free of cells that exhibit an exhausted, anergic, and/or regulatory T cell phenotype.

In embodiments, the VSTs comprise both effector memory T cells and central memory T cells. Thus, in embodiments, the VSTs comprise cells with a CD45RO+/CD62L+ (central memory) phenotype as well as cells with a CD45RO+/CD62L- (effector memory) phenotype. In embodiments, the VSTs comprise at least at least about 10%, at least about 20%, at least about 30%, at least about 40%, at least about 50%, at least about 60%, at least about 70%, or more T cells with a central memory phenotype. In embodiments, the VSTs comprise at least at least about 10%, at least about 20%, at least about 30%, at least about 40%, at least about 50%, at least about 60%, at least about 70%, or more T cells with an effector memory phenotype.

In some embodiments, the VSTs are Th1 polarized. In some embodiments, the VSTs are predominantly Th1 polarized. In some embodiments, the VSTs comprise both CD4+ and CD8+ T cells. In some embodiments, the VSTs are reactive against SARS-CoV2. In some embodiments, the VSTs produce IFNγ and/or TNFα. In some embodiments, the VSTs produce both IFNγ and TNFα. In some embodiments, the majority of VSTs that produce IFNγ also produce TNFα. In some embodiments, the VSTs produce one or more effector molecules selected from Granzyme B, IFNγ, TNFα, MIP-1α, and perforin. In some embodiments, the VSTs produce one or more chemoattractive molecule, e.g., MIP-1β. In some embodiments, the VSTs are polyfunctional. As used herein, the term “polyfunctional” refers to T cells that are capable of more than one effector function. Effector functions include production of one or more cytokines and/or chemokines, and lysis of target cells, For example, in some embodiments, the VSTs produce two or more (e.g., 3, 4, 5, or more) different effector molecules. In some embodiments, at least about 5%, at least about 10%, at least about 15%, at least about 20%, at least about 25%, at least about 50%, or more of the VSTs are polyfunctional. In some embodiments, at least about 5%, at least about 10%, at least about 15%, at least about 20%, at least about 25%, or more of the CD4+ T cells in the VST population are polyfunctional. In some embodiments, at least about 2%, at least about 4%, at least about 5%, at least about 10%, at least about 15%, at least about 20%, at least about 25%, or more of the CD8+ T cells in the VST population are polyfunctional.

In some embodiments, the VSTs are able to lyse viral antigen-expressing targets cells. In some embodiments, the VSTs are able to lyse other suitable types of antigen-expressing targets cells. In some embodiments, the VSTs in the compositions do not significantly lyse non-infected autologous target cells. In some embodiments, the VSTs in the compositions do not significantly lyse non-infected autologous allogenic target cells.

The present disclosure provides pharmaceutical compositions comprising any compositions formulated for intravenous delivery. In some embodiments, the compositions are negative for bacteria for at least 1, at least 2, at least 3, at least 4, at least 5, at least 6, at least 7, at least 8 days, at least 9 days, at least 10 days, in culture. In some embodiments, the compositions are negative for bacteria for at least 7 days in culture. In some embodiments, the compositions are negative for fungi for at least 1, at least 2, at least 3, at least 4, at least 5, at least 6, at least 7, at least 8 days, at least 9 days, at least 10 days, in culture. In some embodiments, the compositions are negative for fungi for at least 7 days in culture.

The present pharmaceutical compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the present pharmaceutical compositions are negative for mycoplasma.

The present disclosure provides methods of lysing a target cell comprising contacting the target cell with the compositions or pharmaceutical compositions as described herein. In some embodiments, the contacting between the target cell and the compositions or pharmaceutical compositions occurs in vivo in a subject. In some embodiments, the contacting between the target cell and the compositions or pharmaceutical compositions occurs in vivo via administration of the VSTs to a subject. In some embodiments, the subject is a human.

The present disclosure provides methods of treating or preventing a viral infection comprising administering to a subject in need thereof the compositions or the pharmaceutical compositions as described herein. In some embodiments, the VSTs between 5x10³ and 5x10⁹ VSTs / m², 5x10⁴ and 5x10⁸ VSTs / m², 5x10⁵ and 5x10⁷ VSTs / m², 5x10⁴ and 5x10⁸ VSTs / m², 5x10⁶ and 5x10⁹ VSTs / m², inclusive of all ranges and subranges therebetween. In some embodiments, the VSTs are administered to the subject. In some embodiments, the subject is immunocompromised. In some embodiments, the subject has acute myeloid leukemia. In some embodiments, the subject has acute lymphoblastic leukemia. In some embodiments, the subject has chronic granulomatous disease.

In some embodiments, the subject can have one or more medical conditions. In some embodiments, the subject receives a matched related donor transplant with reduced intensity conditioning prior to receiving the VSTs. In some embodiments, the subject receives a matched unrelated donor transplant with myeloablative conditioning prior to receiving the VSTs. In some embodiments, the subject receives a haplo-identical transplant with reduced intensity conditioning prior to receiving the VSTs. In some embodiments, the subject receives a matched related donor transplant with myeloablative conditioning prior to receiving the VSTs. In some embodiments, the subject has received a solid organ transplantation. In some embodiments, the subject has received chemotherapy. In some embodiments, the subject has an HIV infection. In some embodiments, the subject has a genetic immunodeficiency. In some embodiments, the subject has received an allogeneic stem cell transplant. In some embodiments, the subject has more than one medical conditions as described in this paragraph. In some embodiments, the subject has all medical conditions as described in this paragraph. In some embodiments, the subject has no other medical conditions other than infection with SARS-CoV2.

In some embodiments, the composition as described herein is administered to the subject a plurality of times. In some embodiments, the composition as described herein is administered to the subject more than one time. In some embodiments, the composition as described herein is administered to the subject more than two times. In some embodiments, the composition as described herein is administered to the subject more than three times. In some embodiments, the composition as described herein is administered to the subject more than four times. In some embodiments, the composition as described herein is administered to the subject more than five times. In some embodiments, the composition as described herein is administered to the subject more than six times. In some embodiments, the composition as described herein is administered to the subject more than seven times. In some embodiments, the composition as described herein is administered to the subject more than eight times. In some embodiments, the composition as described herein is administered to the subject more than nine times. In some embodiments, the composition as described herein is administered to the subject more than ten times. In some embodiments, the composition as described herein is administered to the subject a number of times that are suitable for the subjects.

In some embodiments, the administration of the composition effectively treats or prevents a viral infection in the subject. In some embodiments, the viral infection is PIV. In some embodiments, the viral infection is RSV. In some embodiments, the viral infection is Influenza. In some embodiments, the viral infection is hMPV. In some embodiments the viral infection is SARS-CoV2. In some embodiments the viral infection is SARS-CoV. In some embodiments the viral infection is MERS-CoV. In some embodiments the viral infection is HCoV-HKU1. In some embodiments the viral infection is, and HCoV-OC43. In some embodiments the viral infection is HCoV-E229. In some embodiments the viral infection is HCoV-NL63.

The present disclosure provides compositions comprising a polyclonal population of VSTs that recognize a plurality of viral antigens. The present disclosure provides that the plurality of viral antigens comprise at least one antigen. In some embodiments, the at least one antigen can be SARS-CoV2. In some embodiments, the at least one antigen can be from PIV. In some embodiments, the at least one antigen can be an RSV antigen. In some embodiments, the at least one antigen can be from Influenza. In some embodiments, the at least one antigen can be from hMPV.

In some embodiments, the present disclosure provides a polyclonal population of VSTs that recognize a plurality of viral antigens comprising at least one antigen from each of PIV, RSV, Influenza, and hMPV. In some embodiments, the present disclosure provides a polyclonal population of VSTs that recognize a plurality of viral antigens comprising the plurality of viral antigens comprise at least two antigens from each of PIV, RSV, Influenza, and hMPV.

In some embodiments, the plurality of antigens comprise PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens can be selected from any of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N.

In some embodiments, the present disclosure provides pharmaceutical compositions comprising the compositions as described herein formulated for intravenous delivery. In some embodiments, the composition as described herein is negative for bacteria. In some embodiments, the composition as described herein is negative for fungi. In some embodiments, the composition as described herein is negative for bacteria or fungi for at least 1 days, at least 2 days, at least 3 days, at least 4 days, at least 5 days, at least 6 days, at least 7 days, at least 8 days, at least 9 days, at least 10 days, in culture. In some embodiments, the composition as described herein is negative for bacteria or fungi for at least 7 days in culture.

In some embodiments, the pharmaceutical compositions formulated for intravenous delivery exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the pharmaceutical compositions formulated for intravenous delivery are negative for mycoplasma.

### EXAMPLES

### Example 1 Identification of SARS-CoV2 Antigens

The choice of antigens to explore as potential immunogenic targets in SARS-CoV2 was based on a number of factors including:
(i) Role in viral “fitness” (i.e. viral replication, host immune evasion, target cell infection, etc) - we focused on antigens that were most crucial for virus survival thereby identifying targets that could be optimally exploited by the immune system in combating the virus
(ii) homology with other coronaviruses - this allowed us to simultaneously exploring the potential of developing a T cell therapy that was active not just against SARS-CoV2 but also other coronavirus, should the induced VSTs recognize regions of the antigens that were conserved in other serotypes and strains.

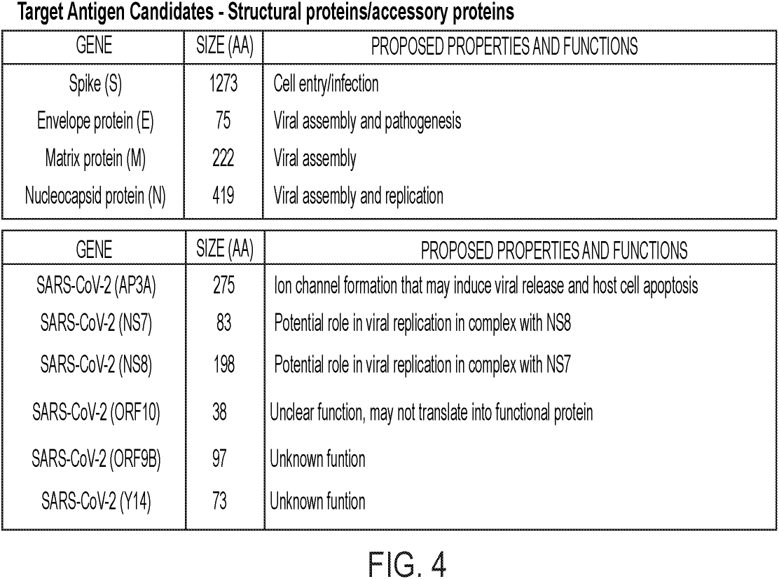

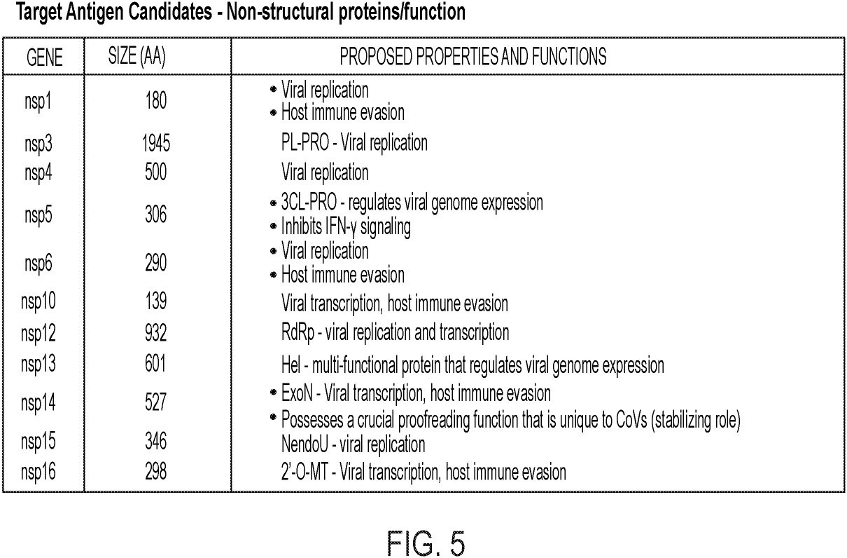

Based on these parameters, we chose the following SARS-CoV2 antigens for further immunogenicity analysis: nsp1; nsp3; nsp4; nsp5; nsp6; nsp7; nsp8 nsp10; nsp12; nsp13; nsp14; nsp15; and nsp16; Spike (S); Envelope protein (E); Matrix protein (M); and Nucleocapsid protein (N). We also chose to analyze SARS-CoV-2 (AP3A); SARS-CoV-2 (ORF10); SARS-CoV-2 (ORF9B); and SARS-CoV-2 (Y14). The initially elected target antigens and their size / functions are shown in FIG. 4 and FIG. 5.

Pepmixes covering these antigens were ordered (JPT and Genemed Synthesis, Inc) and PBMCs are stimulated according to the following protocol.

For manufacturing, blood is collected and PBMCs are isolated. PBMCs are seeded in a G-Rex 5 bioreactor (Wilson Wolf, Minneapolis, MN) and cultured in TexMACs medium, containing IL4, IL7 and SARS-CoV2 pepmixes (2 ng/peptide/ml) Post stimulation and expansion, T cells are harvested, for cryopreservation and evaluated for virus specificity by IFN_{γ} enzyme-linked immunospot (ELISpot) assay.

VSTs are administered allogeneically to patients with SARS-CoV2 infection or to patients diagnosed with COVID-19. Each patient receives a single intravenous infusion of one or a combination of third party VSTs with the option to receive additional infusions. Therapy with standard antiviral medications may be administered at the discretion of the treating physician.

SARS-CoV2 loads in nasal swab samples are monitored by quantitative PCR (qPCR) in Clinical Laboratory Improvement Amendments (CLIA)-approved laboratories.

### Example 2. Preparation and Characterization of VSTs Specific for SARS-CoV2

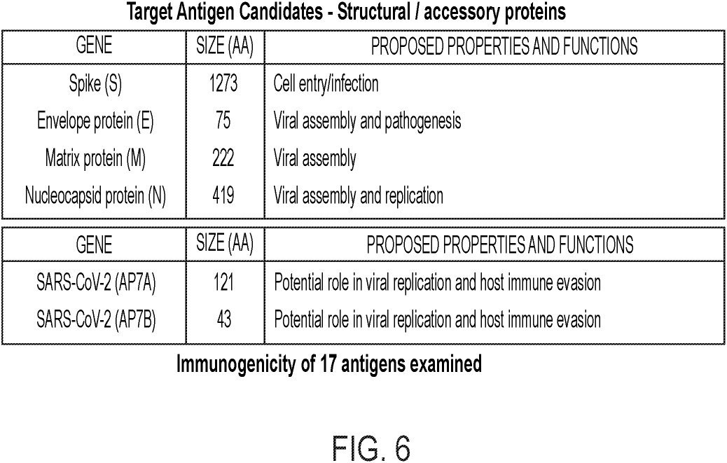

FIGS. 6-12 show the strategy for activating and expanding VSTs specific for SARS-CoV2, and characterization of the expanded VSTs. One exemplary set of candidate antigens (revised in relation to the initial analysis strategy shown in FIG. 4) is provided in FIG. 6.

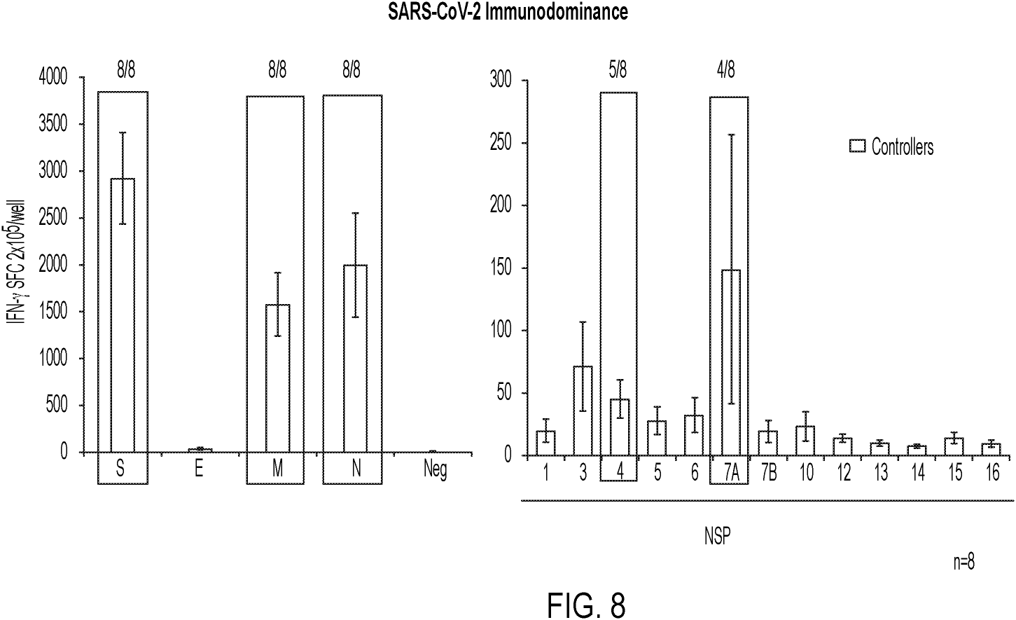

To identify immunodominant antigens, PBMCs from convalescent individuals with mild COVID-19 (not requiring hospitalization) were screened for T cell activity against overlapping peptide libraries (pepmixes) spanning 17 structural and non-structural SARS-COV-2 proteins. As shown in FIG. 8, Spike (S), Matrix (M), Nucleoprotein (N), non-structural protein 3 (nsp3), and non-structural accessory protein 7A (AP7A) were identified as immunodominant (FIG. 8).

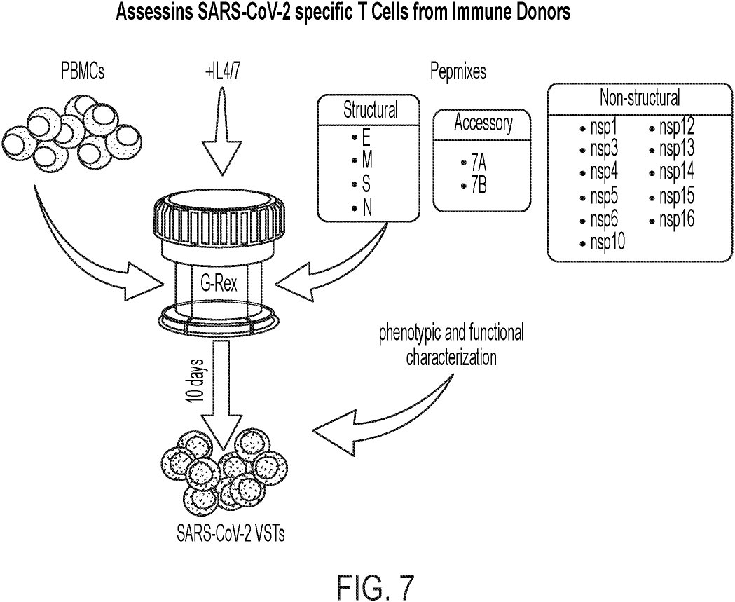

FIG. 7 provides a schematic depiction of the VST cell line production process. PBMCs are isolated from blood and seeded into a G-Rex 5 bioreactor and cultured in medium supplemented with IL4, IL7, and pepmixes. After 10 days of culture, SARS-CoV-2 VST were collected and phenotypically and functionally characterized.

In one study, 8 antigens were used as stimulating antigens (S, M, N, nsp3, nsp4, nsp6, nsp12, and AP7A) and a mean 29±7 fold expansion (mean±SEM; n=5) of cells was achieved. The cells were comprised almost exclusively of CD3+ T cells (97.1±0.7%; mean±SEM), with a mixture of cytotoxic (CD8+; 10.2±1.2%) and helper (CD4+; 85.5±1.8%) T cells. To confirm the anti-viral activity of the expanded cells, an IFNγ ELIspot using each of the individual stimulating antigens as an immunogen. All lines proved to be reactive against the target antigens [S: 2,118±479 SFC/2x10⁵; M: 1,084±182; N: 1,124±335; Nsp3: 71±48.6; Nsp4: 68±30; Nsp6: 23±6.7; AP7a: 65±43; and Nsp12: 29±9]. The VSTs were predominantly Th1-polarized and polyfunctional, producing TNFα, GM-CSF and Granzyme B, as assessed by single-cell protein analysis.

In another study, S, M, N, nsp4, and AP7a were added to the IL4 and IL7 supplemented media and cultured with PBMS in the G-Rex. A mean 9.3 +1.1 fold expansion (mean+SEM; n=8) was achieved with a single stimulation. The anti-viral activity of the expanded cells was tested in an IFNγ ELIspot using each of the individual stimulating antigens as an immunogen and all lines proved to be reactive against the target antigens (S: 2,926±487 SFC/2x10⁵; M: 1,579±339; N: 1,999±556; Nsp4: 45±15; and AP7a: 149±108).

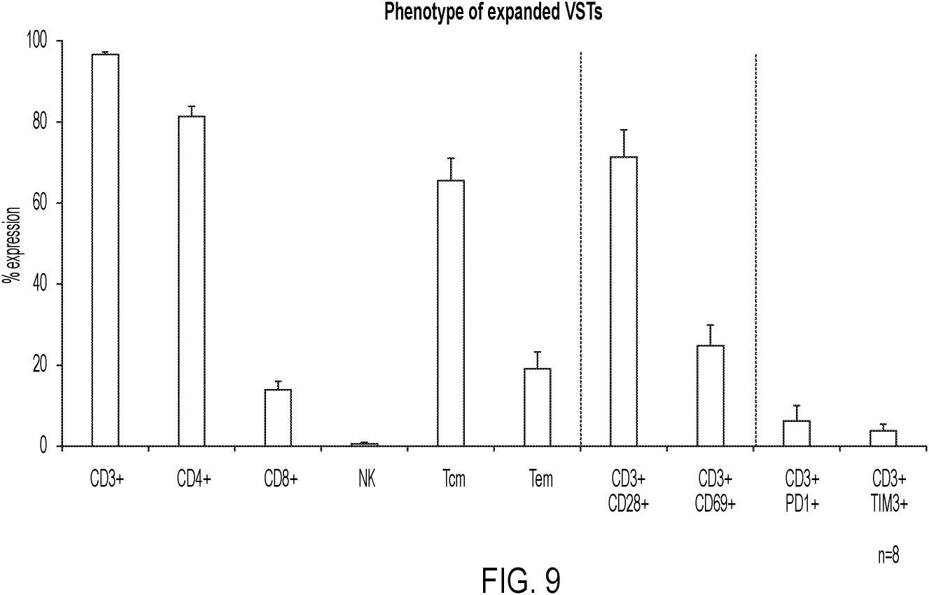

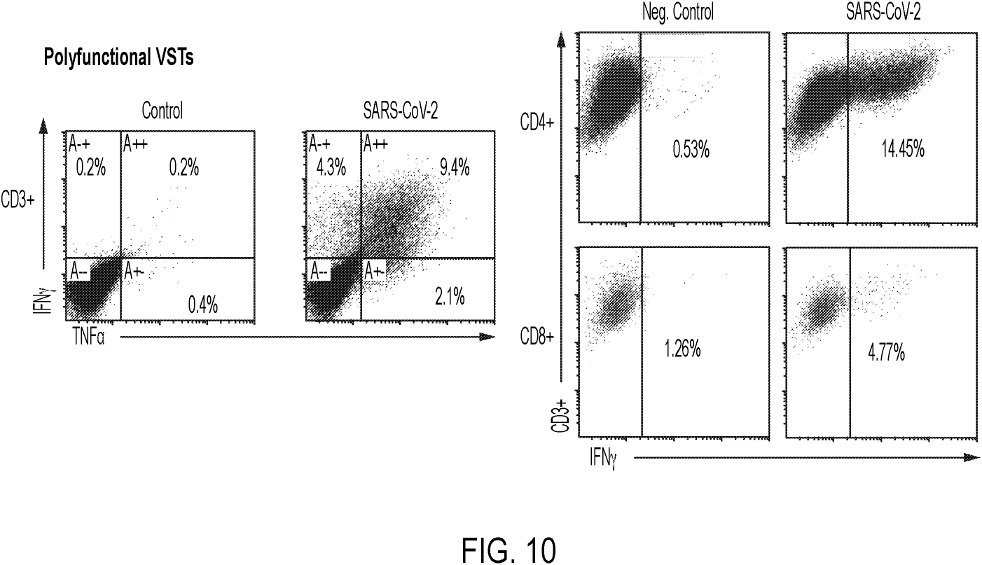

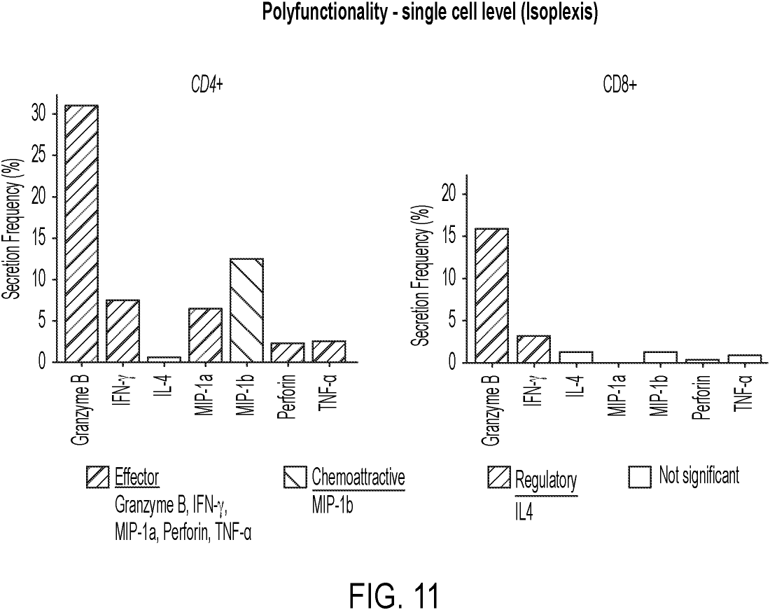

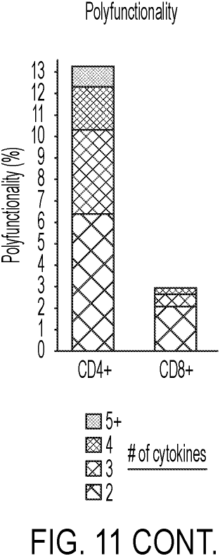

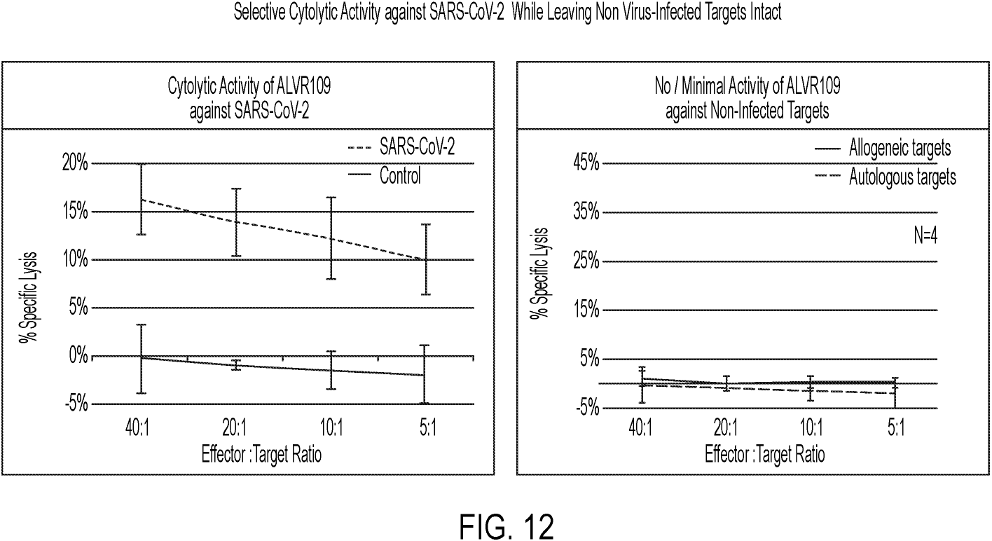

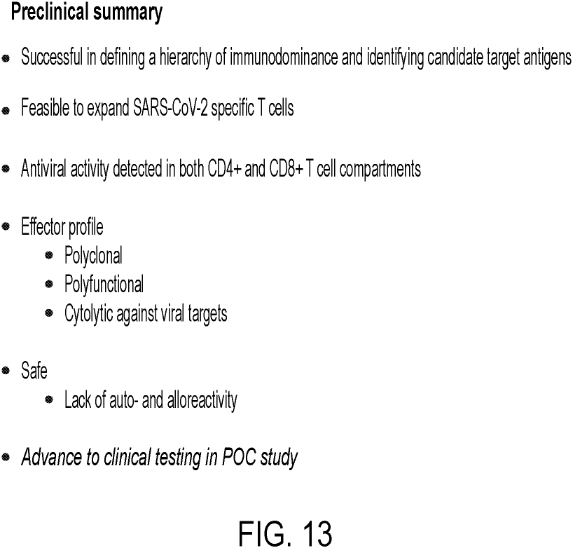

The cells were comprised almost exclusively of CD3+ T cells (97±0.5%), with a mixture of cytotoxic (CD8+) and helper (CD4+) T cells that expressed the activation markers CD69 and CD28 as well as central (CD45RO+/CD62L+) and effector memory markers (CD45RO+/CD62L-), with minimal PD1 or Tim3 expression (FIG. 9). As demonstrated by intracellular cytokine staining (ICS), the cells were responsive against SARS-CoV-2 and the response was mediated by both CD4+ and CD8+ T cell subsets, and the majority of IFNy-producing cells also produced TNFα (FIG. 10). Reactive cells exhibited a primarily Th1-polarized profile as measured by Granzyme B production, luminex array and single-cell protein analysis (FIG. 11). In addition, the expanded cells were able to kill viral pepmix-loaded autologous PHA blasts with minimal/no activity against non-antigen-expressing autologous and allogeneic targets (FIG. 12).

In summary, the data showed that SARS-CoV2 specific T cells could be expanded using the indicated method, that the expanded cells included both CD4+ and CD8+ T cells that were polyclonal, polyfunctional, and cytolytic. For example, the data showed that SARS-CoV2 specific T cells specific for target antigens S, M, N, nsp4, and AP7A were effectively expanded and the expanded cells were polyclonal, polyfunctional, and cytolytic.

Further, the SARS-CoV2 specific T cells lacked off-target activity and lacked auto- and allo-reactivity. Accordingly, the data showed that the expanded SARS-CoV2 specific T cells were suitable to advance to clinical testing.

### Example 3. Clinical Study

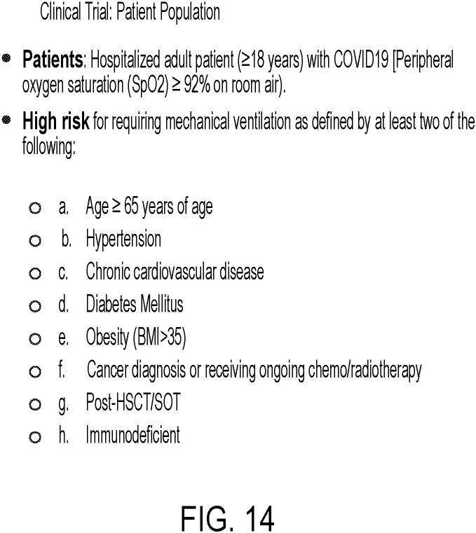

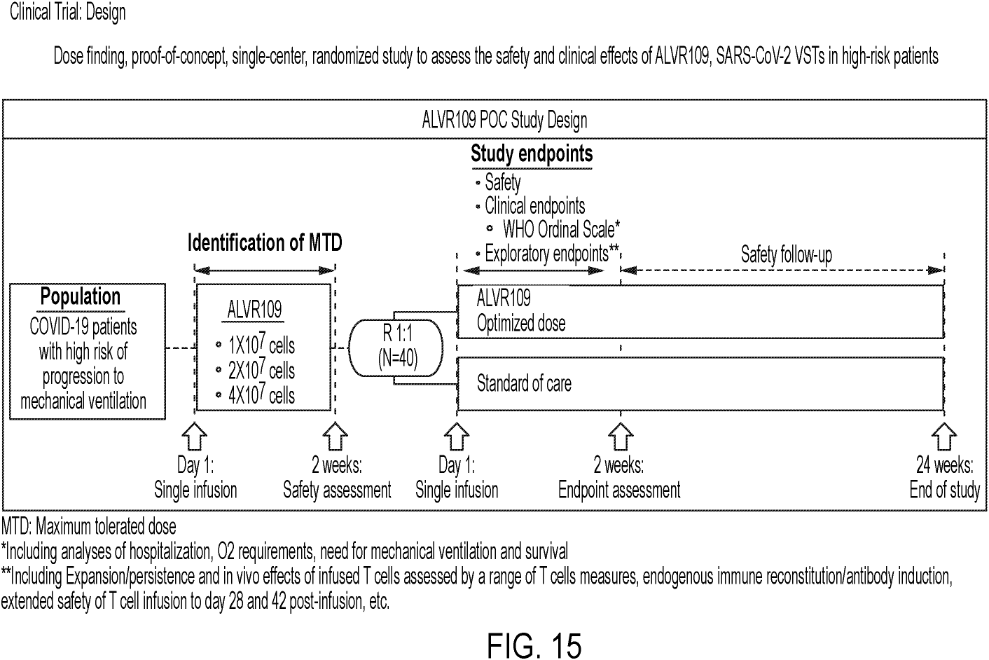

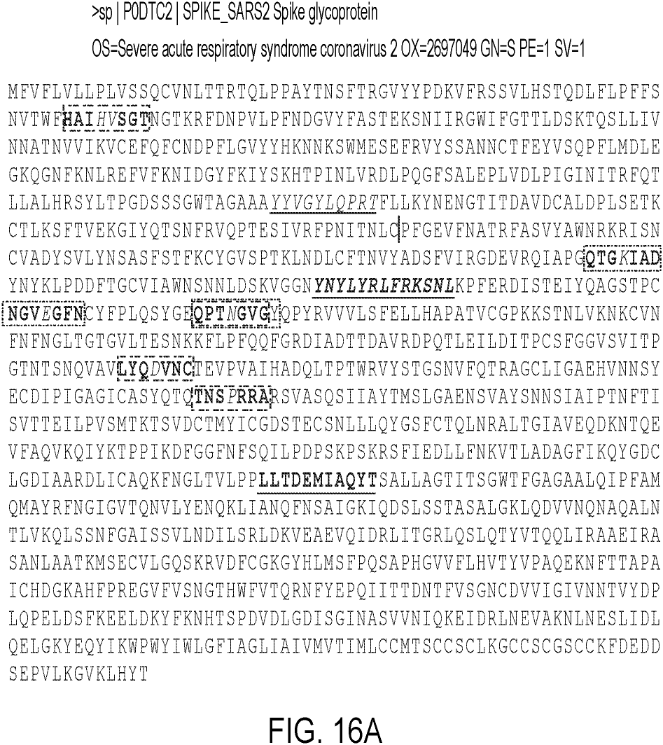

A study is conducted to assess the safety and clinical effects of the SARS-CoV2 VSTs. An exemplary patient population is provided in FIG. 14. An exemplary study design is shown in FIG. 15. whereby the maximal tolerated dose is established in a run-in dose escalation study with increasing cell doses administered to successive patients. Examples of the infused cell doses include 1 × 107, 2 × 107, or 4 × 10⁷ VSTs to COVID-19 patients at high risk of progression to mechanical ventilation. After infusion, safety and clinical endpoints are monitored in patients that received the VSTs compared to standard of care. Clinical endpoints include analyses of hospitalization, oxygen requirements, need for mechanical ventilation, and survival. Exploratory endpoints include expansion/persistence and in vivo effects of infused T cells assessed by a range of T cell measures, endogenous immune reconstitution/antibody induction, and extended safety of T cell infusion to day 28 and 42 post-infusion.

Patients were dosed with 1 × 10⁷ SARS-CoV2 VSTs in the study described above in FIG. 15. The patients in the study were COVID-19 patients with a high risk of progression to mechanical ventilation. Cellular immunity against SARS-CoV-2 antigens targeted by the infused products was tested for each patient pre-VST dosing and on day 14 (±2 days) after VST dosing by IFNγ ELISPOT. An increase in cellular immunity as measured by activity against the target antigens S, M, N, AP7a, and/or nsp4 was observed. The clinical study is ongoing (NCT04401410).

### Example 4. Universal SARS-CoV-2 Specific T Cell Product (UVST)

A pre-clinical study was conducted to assess safety and efficacy of pooled SARS-CoV-2-specific T cell product from distinct HLA type donors, to determine if a SARS-CoV-2 specific T cell product can be generated for universal administration. Efficacy of the pooled universal product (UVST) was assessed by measuring specificity of the UVSTs as compared to specificity of individual donor products. Safety of the UVSTs was assessed by testing for alloreactivity.

Three cell lines with specificity for SARS-CoV-2 were manufactured as described above, for three donors with the disparate HLA types shown below in Table 1.

TABLE 1

Donor HLA Types
HLA

| | A | B | DR | DQ |
|---|---|---|---|---|
| Donor 1 | 02,24 | 35,39 | 4,7 | 9,11 |
| Donor 2 | 01,03 | 37,37 | 06,06 | 10,15 |
| Donor 3 | 11,30 | 15,40 | 2,3 | 8,14 |

Unique immunogenic HLA-restricted epitopes (UE) were identified for each donor to facilitate tracking of each line after pooling (i.e., in the UVST).
UE1SPIKE: Epitope peptide 67 (HLA-2 restricted - unique to Donor 1)
UE2 - SPIKE: Epitope peptide 217 (HLA-A1 restricted - unique to Donor 2)
UE3 - SPIKE: Epitope peptide 113 (HLA-A11 restricted - unique to Donor 3)

The individual SARS-COV-2 specific T cell products from each donor were cryopreserved.

After cryopreservation, a vial from each of the 3 donors was thawed and rested overnight. Cells were re-suspended in VST medium supplemented with cytokines (IL7 at 10 ng/mL and IL4 at 400 U/mL) and transferred to a 24-well plate for overnight incubation at 37° C., 5% CO2.

Post rest, VSTs from each donor (5 x 10⁶ each) were pooled together for a total of 15 x10⁶ VSTs/vial and cryopreserved as a combined universal product (UVST). The UVSTs were then cryopreserved.

Vials of pooled UVST product and the individual VSTs were thawed for potency assessment by IFNγ ELISPOT and for alloreactivity by chromium release assay. The IFNγ ELISPOT was used to evaluate potency against the following structural and non-structural antigens and unique epitope peptides, and controls:
Antigens: SPIKE, MEMBRANE, NEUCLEOCAPSID, NSP4, and AP7A
Unique (tracking) epitope peptides: UE1-67, UE2-217, UE3-113
Positive control: PHA
Negative control: medium only

Spot forming units (SFUs)/2x10⁵ VSTs/well were quantitated using Mabtech IRIS reader. The results are provided below in Table 2. The results demonstrated that UVSTs are potent post-thaw. The identity of each of the individual VST lines was confirmed in the UVSTs.

TABLE 2

Potency and Identity Results

| | | | | UVSTs |
|---|---|---|---|---|
| SPIKE | | | | 5708 |
| MEMBRANE | | | | 1146 |
| NUCLEOCAPSID | 962 | | | |
| NSP 4 | 54 | | | |
| AP 7A | 41 | | | |
| | UVST | Donor 1 | Donor 2 | Donor 3 |
| Donor 1 UE (67) | 623 | 675 | 1 | 11 |
| Donor 2 UE (217) | 475 | 6 | 515 | 5 |
| Donor 3 UE (113) | 1555 | 34 | 98 | 1087 |
| Negative Control | 0 | 3 | 0 | 0 |
| Positive Control | 4226 | 1106 | 1750 | 1205 |

A chromium release assay was performed to assess auto-and allo-reactivity of UVSTs. Effector cells in the assay were the UVSTs; target cells were autologous PHA blasts (Donors 1 and 3) or unrelated donor PHA blasts (Donors 4 and 5). The HLA types of each of these donors are provided below in Table 3.

TABLE 3

Donor HLAs including unrelated donors
HLA

| | A | B | DR | DQ |
|---|---|---|---|---|
| Donor 1 | 02,24 | 35,39 | 4,7 | 9,11 |
| Donor 2 | 01,03 | 37,37 | 06,06 | 10,15 |
| Donor 4 | 24,24 | 40,45 | 3,16 | 10,16 |
| Donor 5 | 24,68 | 07,07 | 07,07 | 15,15 |

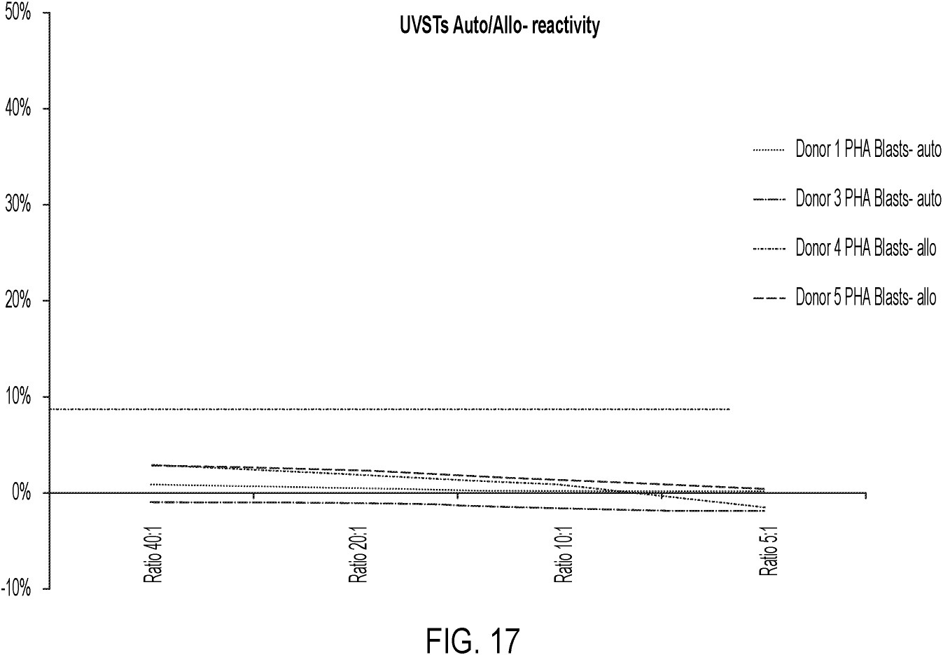

The results of the study are provided in FIG. 17, and demonstrated that the universal SARS-CoV-2 specific T cell product lacked auto- and allo-reactivity.

### Example 5. Reactivity of SARS-CoV-2-specific T Cell Lines Against Peptides Spanning Variant Regions of Variant Strains

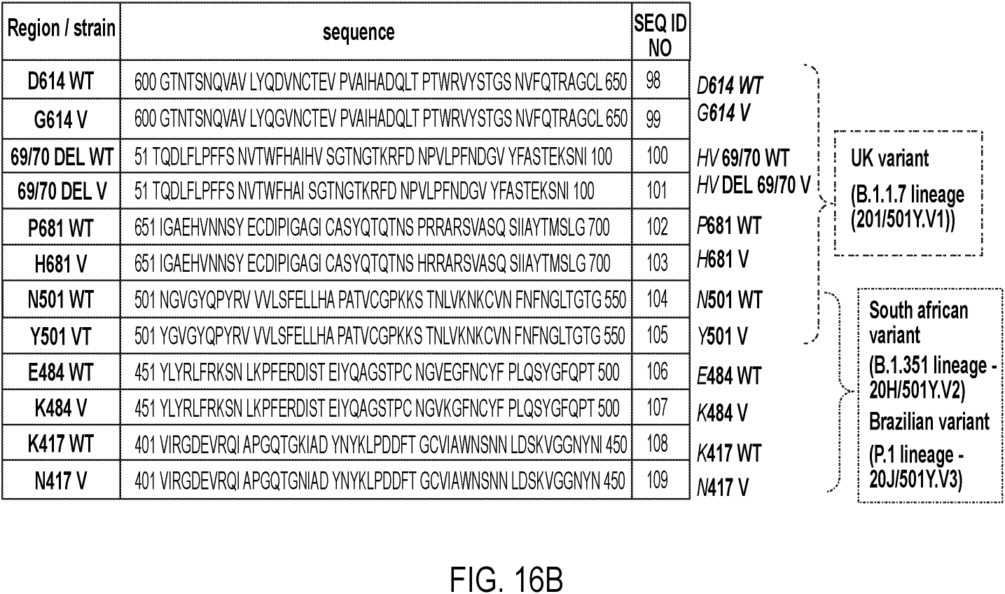

A study was conducted to determine if the SARS-CoV-2-specific T cell lines generated as described above (i.e., using pepmixes spanning the S, N, M, NSP4, and AP7 antigens) demonstrate reactivity against peptides spanning the variant regions of variant strains of SARS-CoV-2 alongside peptides spanning the corresponding sequences of the parental (wild type) strain. The parental (wild type) strain referenced here is the P0DTC2 strain. FIG. 16A shows the Spike protein of the P0DTC2 strain, with boxes around regions of the protein that are mutated in the UK variant (B.1.1.7 lineage - 201/501Y.v1), the South African variant (B.1.352 lineage -20H/501Y.V2), and/or the Brazilian variant P.1 lineage - 201/501Y.V3). The individual WT and mutated region sequences are also provided in FIG. 16B.

In this study, IFNγ ELISPOT was used to evaluate the potency of SARS-CoV-2 VSTs against the stimulating antigen sequences spanning S, M, N, AP7a, NSP4 (p0DTC2 strain), individual specific, unique immunogenic epitope (UIE) peptides within S, and peptides spanning variant regions as well as their wild-type (parental) equivalents.

SARS-CoV-2 VSTs were generated as described above from three donors using pepmixes spanning Spike (S), Membrane (M), Nucleocapsid (N), AP7a, NSP4 based on the p0DTC2 strain sequence), and cryopreserved. Subsequently, the SARS-CoV-2 VSTs were thawed and rested overnight in VST medium supplemented with cytokines (10 ng/mL IL-7 and 400 ng/mL IL-4), and transferred to a 24-well plate for overnight incubation at 37° C., 5% CO₂. The donor HLAs are shown in Table 4.

TABLE 4

Donor HLAs
HLA

| | A | B | DR |
|---|---|---|---|
| Donor 1 | 02,26 | 44,50 | 04,07 |
| Donor 2 | 24,68 | 7,7 | 15,15 |
| Donor 3 | 11,30 | 15,40 | 8,14 |

The UIE peptides were included as positive controls, and are HLA-restricted epitopes with in the S protein derived from regions that were not mutated in the variant strains. The UIE utilized for each donor are shown below, and also shown as bold, underlined text in FIG. 16A.
UIE1 - SPIKE: Epitope peptide YYV (HLA-A2-restricted - unique to Donor 1)
UIE2 - SPIKE: Epitope peptide LLT (HLA-DR15-restricted - unique to Donor 2)
UIE3 - SPIKE: Epitope peptide YNY (HLA-A11-restricted - unique to Donor 3)

To test activity against the variants, 15’mer peptides overlapping by 11 amino acids spanning mutated regions were generated and pooled - (6 variant PepMixes). As a control, 15’mer peptides spanning the corresponding regions of the wildtype/parental strain were also generated - (6 wildtype PepMixes). Antigen-specific activity against N, M, NSP4, and AP7 (p0DTC2 strain) was also assessed and used as a positive control. Table 4 provides the amino acid sequences of each of the S peptides (wild type and variant) used in the study.

TABLE 5

Amino acid sequences of WT and variant peptides

| Strain | Peptide ID | Amino Acid Sequence | SEQ ID NO |
|---|---|---|---|
| Wild Type | D614WT.1 | YQDVNCTEVPVAIHA | 1 |
| | D614WT.2 | VAVLYQDVNCTEVPV | 2 |
| | D614WT.3 | TSNQVAVLYQDVNCT | 3 |
| | D614WT.4 | PGTNTSNQVAVLYQD | 4 |
| | D614WT.5 | DVNCTEVPVAIHADQ | 5 |
| | D614WT.6 | VLYQDVNCTEVPVAI | 6 |
| | D614WT.7 | NQVAVLYQDVNCTEV | 7 |
| | D614WT.8 | TNTSNQVAVLYQDVN | 8 |
| D614G variant | G614V.1 | YQGVNCTEVPVAIHA | 9 |
| | G614V.2 | VAVLYQGVNCTEVPV | 10 |
| | G614V.3 | TSNQVAVLYQGVNCT | 11 |
| | G614V.4 | PGTNTSNQVAVLYQG | 12 |
| | G614V.5 | GVNCTEVPVAIHADQ | 13 |
| | G614V.6 | VLYQGVNCTEVPVAI | 14 |
| | G614V.7 | NQVAVLYQGVNCTEV | 15 |
| | G614V.8 | TNTSNQVAVLYQGVN | 16 |
| Wild Type | 69/70delWT.1 | HVSGTNGTKRFDNPV | 17 |
| | 69/70delWT.2 | FHAIHVSGTNGTKRF | 18 |
| | 69/70delWT.3 | NVTWFHAIHVSGTNG | 19 |
| | 69/70delWT.4 | PFFSNVTWFHAIHVS | 20 |
| | 69/70delWT.5 | IHVSGTNGTKRFDNP | 21 |
| | 69/70delWT.6 | WFHAIHVSGTNGTKR | 22 |
| | 69/70delWT.7 | SNVTWFHAIHVSGTN | 23 |
| | 69/70delWT.8 | LPFFSNVTWFHAIHV | 24 |
| UK variant – B.1.1.7 lineage (201/501Y.V1) | 69/70delV.1 | SGTNGTKRFDNPVLP | 25 |
| | 69/70delV.2 | FHAISGTNGTKRFDN | 26 |
| | 69/70delV.3 | NVTWFHAISGTNGTK | 27 |
| | 69/70delV.4 | PFFSNVTWFHAISGT | 28 |
| | 69/70delV.5 | ISGTNGTKRFDNPVL | 29 |
| | 69/70delV.6 | WFHAISGTNGTKRFD | 30 |
| | 69/70delV.7 | SNVTWFHAISGTNGT | 31 |
| | 69/70delV.8 | LPFFSNVTWFHAISG | 32 |
| Wild Type | P681WT.1 | NSPRRARSVASQSII | 33 |
| | P681WT.2 | QTQTNSPRRARSVAS | 34 |
| | P681WT.3 | CASYQTQTNSPRRAR | 35 |
| | P681WT.4 | GAGICASYQTQTNSP | 36 |
| | P681WT.5 | PRRARSVASQSIIAY | 37 |
| | P681WT.6 | QTNSPRRARSVASQS | 38 |
| | P681WT.7 | SYQTQTNSPRRARSV | 39 |
| | P681WT.8 | GICASYQTQTNSPRR | 40 |
| UK Variant | H681V.1 | NSHRRARSVASQSII | 41 |
| | H681V.2 | QTQTNSHRRARSVAS | 42 |
| | H681V.3 | CASYQTQTNSHRRAR | 43 |
| | H681V.4 | GAGICASYQTQTNSH | 44 |
| | H681V.5 | HRRARSVASQSIIAY | 45 |
| | H681V.6 | QTNSHRRARSVASQS | 46 |
| | H681V.7 | SYQTQTNSHRRARSV | 47 |
| | H681V.8 | GICASYQTQTNSHRR | 48 |
| Wild Type | N501WT.1 | PTNGVGYQPYRVVVL | 49 |
| | N501WT.2 | YGFQPTNGVGYQPYR | 50 |
| | N501WT.3 | PLQSYGFQPTNGVGY | 51 |
| | N501WT.4 | NCYFPLQSYGFQPTN | 52 |
| | N501WT.5 | NGVGYQPYRVVVLSF | 53 |
| | N501WT.6 | FQPTNGVGYQPYRVV | 54 |
| | N501WT.7 | QSYGFQPTNGVGYQP | 55 |
| | N501WT.8 | YFPLQSYGFQPTNGV | 56 |
| UK variant & South African variant (B.1.351 lineage – 20H/501Y.V2) & Brazilian variant (P.1 lineage – 20J/501Y.V3) | Y501V.1 | PTYGVGYQPYRVVVL | 57 |
| | Y501V.2 | YGFQPTYGVGYQPYR | 58 |
| | Y501V.3 | PLQSYGFQPTYGVGY | 59 |
| | Y501V.4 | NCYFPLQSYGFQPTY | 6084 |
| | Y501V.5 | YGVGYQPYRVVVLSF | 6185 |
| | Y501V.6 | FQPTYGVGYQPYRVV | 6286 |
| | Y501V.7 | QSYGFQPTYGVGYQP | 6387 |
| | Y501V.8 | YFPLQSYGFQPTYGV | 64 |
| Wild Type | E484WT.1 | GVEGFNCYFPLQSYG | 65 |
| | E484WT.2 | TPCNGVEGFNCYFPL | 66 |
| | E484WT.3 | QAGSTPCNGVEGFNC | 67 |
| | E484WT.4 | TEIYQAGSTPCNGVE | 68 |
| | E484WT.5 | EGFNCYFPLQSYGFQ | 69 |
| | E484WT.6 | CNGVEGFNCYFPLQS | 70 |
| | E484WT.7 | GSTPCNGVEGFNCYF | 71 |
| | E484WT.8 | IYQAGSTPCNGVEGF | 72 |
| South African variant & Brazilian variant | K484V.1 | GVKGFNCYFPLQSYG | 73 |
| | K484V.2 | TPCNGVKGFNCYFPL | 74 |
| | K484V.3 | QAGSTPCNGVKGFNC | 75 |
| | K484V.4 | TEIYQAGSTPCNGVK | 76 |
| | K484V.5 | KGFNCYFPLQSYGFQ | 77 |
| | K484V.6 | CNGVKGFNCYFPLQS | 78 |
| | K484V.7 | GSTPCNGVKGFNCYF | 79 |
| | K484V.8 | IYQAGSTPCNGVKGF | 80 |
| Wild Type | K417WT.1 | TGKIADYNYKLPDDF | 81 |
| | K417WT.2 | APGQTGKIADYNYKL | 82 |
| | K417WT.3 | VRQIAPGQTGKIADY | 83 |
| | K417WT.4 | RGDEVRQIAPGQTGK | 84 |
| | K417WT.5 | KIADYNYKLPDDFTG | 85 |
| | K417WT.6 | GQTGKIADYNYKLPD | 86 |
| | K417WT.7 | QIAPGQTGKIADYNY | 87 |
| | K417WT.8 | DEVRQIAPGQTGKIA | 88 |
| South African variant & Brazilian variant | N417V.1 | TGNIADYNYKLPDDF | 89 |
| | N417V.2 | APGQTGNIADYNYKL | 90 |
| | N417V.3 | VRQIAPGQTGNIADY | 91 |
| | N417V.4 | RGDEVRQIAPGQTGN | 92 |
| | N417V.5 | NIADYNYKLPDDFTG | 93 |
| | N417V.6 | GQTGNIADYNYKLPD | 94 |
| | N417V.7 | QIAPGQTGNIADYNY | 95 |
| | N417V.8 | DEVRQIAPGQTGNIA | 96 |

Potency was assessed by IFNγ ELISPOT. Spot Forming Units (SFUs)/2x10⁵ VSTs/well were quantitated (Mabtech IRIS). The results are shown in Table 6.

TABLE 6

Potency Results

| | | Donor 1 | Donor 2 | Donor 3 |
|---|---|---|---|---|
| SPIKE Antigen | | 4438 | 3940 | 4071 |
| | SPIKE: UIEs | 259 | 2257 | 1172 |
| | WT 614 | 19 | 114 | 1 |
| | V614 | 33 | 15 | 10 |
| | WT69/70 | 141 | 628 | 19 |
| | V69/70 | 88 | 440 | 19 |
| | WT681 | 66 | 445 | 12 |
| | V681 | 0 | 42 | 6 |
| | WT501 | 6 | 30 | 42 |
| | V501 | 10 | 45 | 17 |
| | WT484 | 17 | 103 | 11 |
| | V484 | 14 | 18 | 34 |
| | WT417 | 17 | 9 | 24 |
| | V417 | 14 | 14 | 8 |
| M Antigen | | 1867 | 169 | 711 |
| N Antigen | | 2102 | 196 | 823 |
| NSP4 Antigen | | 7 | 44 | 25 |
| AP7A Antigen | | 4 | 11 | 0 |

The study demonstrated that SARS-CoV2-VSTs targeting S, M, N, AP7a, NSP4 are potent, and recognize the S antigen of the parental sequence (p0DTC2 strain) as well as UIEs within SPIKE that are conserved between parental and variant sequences. Further, the results showed that for each variant and its WT equivalent:
Neither the WT or the variant was immunogenic (e.g., WT/V417);
The variant version reduced but did not abrogate potency (e.g., WT/V 69/70 response in Donors 1 and 2); or
Variant peptides were not recognized (WT/V 614 response in Donor 2).

Even in cases where mutated sequences resulted in loss of potency against a single peptide, activity against other immunogenic Spike-derived peptides (UIEs) as well as other structural and non-structural antigens was retained. Therefore, the study demonstrates that SARS-CoV2 VSTs described herein are potent against both parental and variant SARS-CoV-2 viral strains.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A composition comprising a polyclonal population of virus-specific T-lymphocytes (VSTs) that recognize one or more SARS-CoV2 antigens.

2. The composition of claim 1, wherein the VSTs are generated by contacting peripheral blood mononuclear cells (PBMCs) with one or more pepmix libraries, each pepmix library containing a plurality of overlapping peptides spanning all or at least a portion of the SARS-CoV2 antigens.

3. The composition of claims 1, wherein the VSTs are generated by contacting T cells with antigen presenting cells (APCs) such as dendritic cells (DCs) primed with a plurality of pepmix libraries, each pepmix library containing a plurality of overlapping peptides spanning all or at least a portion of the SARS-CoV2 antigens.

4. The composition of claim 1, wherein the VSTs are generated by contacting T cells with APCs such as DCs nucleofected with at least one DNA plasmid encoding at least one or a portion of one SARS-CoV2 antigen.

5. The composition of any one of claims 1-4, wherein the VSTs are cultured ex vivo in the presence of both IL-7 and IL-4.

6. The composition of any one of claims 1-5, wherein the VSTs have expanded sufficiently within 9-18 days of culture such that they are ready for administration to a patient.

7. A composition comprising a polyclonal population of VST that recognize one or more antigens, or a portion thereof, from SARS-CoV2 and one or more additional antigens, or a portion thereof, from one or more additional virus.

8. The composition of claim 7, wherein the additional virus comprises a different coronavirus serotype/strain.

9. The composition of claim 7 or 8, wherein the additional virus comprises a β-coronavirus (β-CoV).

10. The composition of claim 7 or 8, wherein the additional virus comprises an alpha-coronavirus (α-CoV).

11. The composition of claim 9, wherein the additional virus is a β-CoV selected from SARS-CoV, MERS-CoV, HCoV-HKU1, and HCoV-OC43.

12. The composition of claim 10, wherein the additional virus is an α-CoV selected from HCoV-E229 and HCoV-NL63.

13. The composition of any one of claims 7-12, wherein the additional virus comprises another respiratory virus.

14. The composition of claim 13, wherein the respiratory virus is selected from parainfluenza virus (PIV), respiratory syncytial virus (RSV), Influenza, human metapneumovirus (hMPV), adenovirus (AdV), and combinations thereof.

15. The composition of claim 13, wherein the respiratory virus comprises PIV, RSV, Influenza, hMPV, and AdV.

16. The composition of any one of claims 7-15, wherein the VSTs are generated by contacting PBMCs with a plurality of pepmix libraries, each pepmix library containing a plurality of overlapping peptides spanning all or a portion of a SARS-CoV2 antigen or an antigen from the one or more additional viruses.

17. The composition of any one of claims 7-15, wherein the VSTs are generated by contacting T cells with APCs such as DCs primed with a plurality of pepmix libraries, each pepmix library containing a plurality of overlapping peptides spanning all or a portion of a viral antigen, wherein at least one of the plurality of pepmix libraries spans a first antigen from SARS-CoV2 and wherein at least one (or a portion of one) additional pepmix library of the plurality of pepmix libraries spans each second antigen.

18. The composition of any one of claims 7-15, wherein the VSTs are generated by contacting T cells with APCs such as DCs nucleofected with at least one DNA plasmid encoding at least one SARS-CoV2 antigen, or a portion thereof, and at least one DNA plasmid encoding each second antigen, or a portion thereof.

19. The composition of claim 4 or 18, wherein the plasmid encodes at least one SARS-CoV2 antigen, or a portion thereof, and at least one of the additional antigens, or a portion thereof.

20. The composition of any one of claims 1-19, wherein the VSTs comprise CD4+ T-lymphocytes and CD8+ T-lymphocytes.

21. The composition of any one of claims 1-20, wherein the VSTs express αβ T cell receptors.

22. The composition of any one of claims 1-21, comprising MHC-restricted VSTs.

23. The composition of any one of claims 1-21, wherein the VSTs comprise central memory and effector memory T cells.

24. The composition of any one of claims 1-23, wherein the SARS-CoV2 antigen comprises one or more antigens selected from the group consisting of non-structural protein 1(nsp1); nsp3; nsp4; nsp5; nsp6; nsp10; nsp12; nsp13; nsp14; nsp15; nsp16; accessory protein 7A (AP7A) or AP7B.

25. The composition of any one of claims 1-24, wherein the SARS-CoV2 antigen comprises one or more antigen selected from the group consisting of Spike (S); Envelope protein (E); Matrix protein (M); and Nucleocapsid protein (N).

26. The composition of any one of claims 1-24, wherein the SARS-CoV2 antigen comprises one or more antigen (i) selected from the group consisting of SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14) or one or more antigen that is a structural antigen and one or more antigen that is a non-structural antigen.

27. The composition of any one of claims 1-26, wherein the SARS-CoV2 antigens comprise Spike (S), Matrix protein (M), Nucleocapsid protein (N), non-structural protein 4 (nsp4), and accessory protein 7a (AP7A).

28. The composition of claim 27, wherein the SARS-COV2 antigens further comprise nsp3, nsp6, and/or nsp12.

29. The composition of any one of claims 1-28, wherein the SARS-CoV2 antigens consist of S, M, N, nsp4, and AP7A.

30. The composition of any one of claims 7-29, wherein the additional antigen is selected from the group consisting of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, hMPV antigen N, and AdV antigen Hexon, AdV antigen Penton and combinations thereof.

31. The composition of any one of claims 7-30, wherein the additional antigen comprises PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, hMPV antigen N, AdV antigen Hexon, AdV antigen Penton and combinations thereof.

32. The composition of any one of claims 1-31, wherein the VSTs are cultured ex vivo in the presence of both IL-7 and IL-4.

33. The composition of any one of claims 1-32, wherein the VSTs have expanded sufficiently within 9-18 days of culture such that they are ready for administration to a patient.

34. The composition of any one of claims 1-33, wherein the VSTs exhibit one or more property selected from
a. negligible alloreactivity;
b. less activation induced cell death of antigen-specific T cells harvested from a patient than corresponding antigen-specific T cells harvested from the same patient, but not cultured in the presence of both IL-7 and IL-4; and
c. viability of greater than 70%.

35. The composition of any one of claims 1-34, wherein the composition is negative for bacteria and fungi for at least 7 days in culture; exhibit less than 5 EU/ml of endotoxin, and are negative for mycoplasma.

36. The composition of any one of claims 1-35, wherein the pepmixes are chemically synthesized and are >70% pure.

37. The composition of any one of claims 1-36, wherein the VSTs are Th1 polarized.

38. The composition of any one of claims 1-37, wherein the VSTs produce effector cytokines/molecules including IFN-gamma, TNF-alpha, GM-CSF, Granzyme-B, or perforin upon exposure to antigen.

39. The composition of any one of claims 1-38, wherein the VSTs are able to lyse viral antigen-expressing targets cells.

40. The composition of any one of claims 1-39, wherein the VSTs do not significantly lyse non-infected autologous or allogenic target cells.

41. A pharmaceutical composition comprising the composition of any one of claims 1-40 formulated for intravenous delivery, wherein the composition is negative for bacteria and fungi for at least 7 days in culture; exhibit less than 5 EU/ml of endotoxin, and are negative for mycoplasma.

42. A method of lysing a target cell comprising contacting the target cell with the composition of any one of claims 1-40 or the pharmaceutical composition of claim 41.

43. The method of claim 42, wherein the contacting occurs in vivo in a subject.

44. The method of claim 42 or 43, wherein the contacting occurs in vivo via administration of the VSTs to a subject.

45. A method of treating or preventing a viral infection comprising administering to a subject in need thereof the composition of any one of claims 1-40 or the pharmaceutical composition of claim 41 or the universal antigen-specific T cell therapy product of claim 89.

46. The method of any one of claims 43-45, wherein between 5×10⁶ and 5×10⁷ VST/m² are administered to the subject.

47. The method of any one of claims 43-46, wherein the subject is immunocompetent or immunocompromised.

48. The method of any one of the claims 43-47, wherein the subject is infected with SARS-CoV2 or has been diagnosed with COVID-19.

49. The method of any one of claims 43-48, wherein the subject has acute myeloid leukemia, acute lymphoblastic leukemia, or chronic granulomatous disease.

50. The method of any one of claims 43-49, wherein the subject, prior to receiving the VSTs, received:
a. a matched related donor transplant with reduced intensity conditioning;
b. a matched unrelated donor transplant with myeloablative conditioning;
c. a haplo-identical transplant with reduced intensity conditioning; or
d. a matched related donor transplant with myeloablative conditioning.

51. The method of any one of claims 43-50, wherein the subject
a. has received a solid organ transplantation;
b. has received chemotherapy;
c. has an HIV infection;
d. has a genetic immunodeficiency;
e. has received an allogeneic stem cell transplant or an autologous stem cell transplant;
f. has a cardiovascular disease;
g. has diabetes;
h. has a chronic respiratory disease;
i. has hypertension;
j. has cancer
k. has obesity
l. has chronic kidney disease;
m. has Down syndrome;
n. is pregnant;
o. has sickle cell disease; and/or
p. is a smoker.

52. The method of any one of claims 44-51, wherein the composition is administered to the subject a plurality of times.

53. The method of any one of claims 44-52, wherein the administration of the composition effectively treats or prevents a SARS-CoV2 infection in the subject.

54. The method of any one of claims 44-53, wherein the administration of the composition effectively treats or prevents a viral infection in the subject, wherein the viral infection is selected from the group consisting of SARS-CoV2, PIV, RSV, Influenza, hMPV, AdV and a combination thereof.

55. The method of any one of claims 43-54, wherein the subject is a human.

56. A method of treating or preventing a coronavirus infection in a subject comprising administering to the subject a polyclonal population of VSTs generated by a method selected from:
a. contacting PBMCs with one or more pepmix libraries, each pepmix library containing a plurality of overlapping peptides spanning all or at least a portion of one or more SARS-CoV2 antigens;
b. contacting T cells with APCs such as dendritic cells (DCs) primed with a plurality of pepmix libraries, each pepmix library containing a plurality of overlapping peptides spanning all or at least a portion of one or more SARS-CoV2 antigens; or
c. contacting T cells with APCs such as DCs nucleofected with at least one DNA plasmid encoding at least one SARS-CoV2 antigen, or a portion thereof.

57. The method of claim 56, wherein the VSTs are cultured ex vivo in the presence of both IL-7 and IL-4.

58. The method of claim 56 or 57, wherein the VSTs have expanded sufficiently within 9-18 days of culture such that they are ready for administration to a patient.

59. The method of any one of claims 56-58, wherein at least one of the SARS-CoV2 antigens is selected from the group consisting of nsp1; nsp3; nsp4; nsp5; nsp6; nsp10; nsp12; nsp13; nsp14; nsp15; and nsp16.

60. The method of any one of claims 56-59, wherein at least one of the SARS-CoV2 antigens is selected from the group consisting of Spike (S); Envelope protein (E); Matrix protein (M); and Nucleocapsid protein (N).

61. The method of any one of claims 56-60, wherein at least one of the SARS-CoV2 antigens is selected from the group consisting of SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14).

62. The method of any one of claims 56-61, wherein the SARS-CoV2 antigens are selected from the group consisting of nsp1; nsp3; nsp4; nsp5; nsp6; nsp10; nsp12; nsp13; nsp14; nsp15; nsp16; Spike (S); Envelope protein (E); Matrix protein (M); and Nucleocapsid protein (N); SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14), and combinations thereof.

63. The method of any one of claims 56-62, wherein the SARS-CoV2 antigens are selected from S, M, N, nsp4, and AP7A, or a combination thereof.

64. The method of claim 63, wherein the SARS-CoV2 antigens further comprise nsp3, nsp6, and/or nsp12.

65. The composition of any one of claims 56-62, wherein the SARS-CoV2 antigens consist of S, M, N, nsp4, and AP7A.

66. The method of any one of claims 56-65, wherein the coronavirus is a β-coronavirus (β-CoV).

67. The method of any one of claims 56-67, wherein the coronavirus is an alpha-coronavirus (α-CoV).

68. The method of claim 66, wherein the β-CoV is SARS-CoV2.

69. The method of claim 66, wherein the β-CoV is selected from SARS-CoV, MERS-CoV, HCoV-HKU1, and HCoV-OC43.

70. The method of claim 67, wherein the α-CoV is selected from E229 and NL63.

71. The method of any one of claims 66-68, wherein SARs-CoV2 has been detected in the subject.

72. The method of any one of claims 66-68, wherein the subject has been diagnosed with COVID-19.

73. The method of any one of claims 43-72, wherein the subject is over 40 years of age.

74. The method of any one of claims 43-73, wherein the subject is over 60 years of age.

75. The method of any one of claims 43-72, wherein the subject is under 40 years of age.

76. The method of any one of claims 43-75, wherein the subject is at a higher risk of an adverse outcome caused by the coronavirus infection due to a preexisting condition.

77. The method of any one of claims 43-75, wherein the subject is at a higher risk of dying as a result of the coronavirus infection due to a preexisting condition.

78. The method of claim 76 or 77, wherein the preexisting condition is selected from cardiovascular disease, diabetes, chronic respiratory disease, hypertension, cancer, obesity, and a combination thereof.

79. A plurality of compositions, each according to any one of claims 1-40, wherein each composition comprises a polyclonal population of VSTs that differs from one another only in that they were produced from donor PBMTs obtained from different donors.

80. The plurality of compositions of claim 79, wherein none of the donors share all the same HLA alleles.

81. A method according to any one of claims 43-78, wherein the subject is administered with one or a plurality of compositions of claim 79 or 80.

82. The method of claim 81, wherein the plurality of compositions are administered to the subject simultaneously.

83. The method of claim 82, wherein the plurality of compositions are pooled together prior to administration to the subject.

84. The method of claim 83, wherein the pooled compositions are cryopreserved and subsequently thawed prior to administration to the subject.

85. The method of claim 82, wherein the VSTs are not pooled, but are rather administered sequentially to the subject.

86. The method of claim 84, wherein the plurality of compositions are administered to the subject at different times.

87. The method of any one of claims 81-85, wherein the plurality of compositions or sequentially administered VSTs comprise enough HLA variability with respect to one another that greater than 95% of the target patient population will be an HLA match with at least one of the VST lines or with a portion of the plurality of compositions on two or more HLA alleles.

88. The method of any one of claims 56-62, wherein the SARS-CoV2 antigens comprise one or more structural antigen and one or more non-structural antigen.

89. A universal antigen-specific T cell therapy product comprising a plurality of polyclonal populations of virus-specific T cell lymphocytes (VSTs) that recognize one or more SARS-CoV2 antigens, wherein the VSTs are derived from a plurality of different donors, and wherein, optionally, the HLA type of each donor differs from at least one of the other donors on at least two HLA alleles.

90. The universal antigen-specific T cell therapy product of claim 89, wherein the HLA type of each donor differs from at least one of the other donors on at least two HLA alleles.

91. A pharmaceutical composition comprising the universal antigen-specific T cell therapy product of claim 89 or claim 90 formulated for intravenous delivery, wherein the composition is negative for bacteria and fungi for at least 7 days in culture; exhibit less than 5 EU/ml of endotoxin, and is negative for mycoplasma.

92. A method of lysing a target cell comprising contacting target cell with the universal antigen-specific T cell therapy product of claim 89 or claim 90 or the pharmaceutical composition of claim 91.

93. The method of claim 92, wherein the contacting occurs in vivo in a subject.

94. The method of claim 92 or 93, wherein the contacting occurs in vivo via administration of the VSTs to a subject.

95. The method of any one of claims 45-78, 81-88, and 93, wherein the subject is administered the universal antigen-specific T cell therapy product without any knowledge of the subject’s HLA type.


SEQUENCE LISTING (109 sequences)

SEQ ID NO: 1 - protein, 15 aa, Betacoronavirus severe acute respiratory syndrome coronavirus 2
YQDVNCTEVPVAIHA

SEQ ID NO: 2 - protein, 15 aa, Betacoronavirus severe acute respiratory syndrome coronavirus 2
VAVLYQDVNCTEVPV

SEQ ID NO: 3 - protein, 15 aa, Betacoronavirus severe acute respiratory syndrome coronavirus 2
TSNQVAVLYQDVNCT

SEQ ID NO: 4 - protein, 15 aa, Betacoronavirus severe acute respiratory syndrome coronavirus 2
PGTNTSNQVAVLYQD

SEQ ID NO: 5 - protein, 15 aa, Betacoronavirus severe acute respiratory syndrome coronavirus 2
DVNCTEVPVAIHADQ

SEQ ID NO: 6 - protein, 15 aa, Betacoronavirus severe acute respiratory syndrome coronavirus 2
VLYQDVNCTEVPVAI

SEQ ID NO: 7 - protein, 15 aa, Betacoronavirus severe acute respiratory syndrome coronavirus 2
NQVAVLYQDVNCTEV

SEQ ID NO: 8 - protein, 15 aa, Betacoronavirus severe acute respiratory syndrome coronavirus 2
TNTSNQVAVLYQDVN

SEQ ID NO: 9 - protein, 15 aa, Betacoronavirus severe acute respiratory syndrome coronavirus 2
YQGVNCTEVPVAIHA

SEQ ID NO: 10 - protein, 15 aa, Betacoronavirus severe acute respiratory syndrome coronavirus 2
VAVLYQGVNCTEVPV

SEQ ID NO: 11 - protein, 15 aa, Betacoronavirus severe acute respiratory syndrome coronavirus 2
TSNQVAVLYQGVNCT

SEQ ID NO: 12 - protein, 15 aa, Betacoronavirus severe acute respiratory syndrome coronavirus 2
PGTNTSNQVAVLYQG

SEQ ID NO: 13 - protein, 15 aa, Betacoronavirus severe acute respiratory syndrome coronavirus 2
GVNCTEVPVAIHADQ

SEQ ID NO: 14 - protein, 15 aa, Betacoronavirus severe acute respiratory syndrome coronavirus 2
VLYQGVNCTEVPVAI

SEQ ID NO: 15 - protein, 15 aa, Betacoronavirus severe acute respiratory syndrome coronavirus 2
NQVAVLYQGVNCTEV

SEQ ID NO: 16 - protein, 15 aa, Betacoronavirus severe acute respiratory syndrome coronavirus 2
TNTSNQVAVLYQGVN

SEQ ID NO: 17 - protein, 15 aa, Betacoronavirus severe acute respiratory syndrome coronavirus 2
HVSGTNGTKRFDNPV

SEQ ID NO: 18 - protein, 15 aa, Betacoronavirus severe acute respiratory syndrome coronavirus 2
FHAIHVSGTNGTKRF

SEQ ID NO: 19 - protein, 15 aa, Betacoronavirus severe acute respiratory syndrome coronavirus 2
NVTWFHAIHVSGTNG

SEQ ID NO: 20 - protein, 15 aa, Betacoronavirus severe acute respiratory syndrome coronavirus 2
PFFSNVTWFHAIHVS

SEQ ID NO: 21 - protein, 15 aa, Betacoronavirus severe acute respiratory syndrome coronavirus 2
IHVSGTNGTKRFDNP

SEQ ID NO: 22 - protein, 15 aa, Betacoronavirus severe acute respiratory syndrome coronavirus 2
WFHAIHVSGTNGTKR

SEQ ID NO: 23 - protein, 15 aa, Betacoronavirus severe acute respiratory syndrome coronavirus 2
SNVTWFHAIHVSGTN

SEQ ID NO: 24 - protein, 15 aa, Betacoronavirus severe acute respiratory syndrome coronavirus 2
LPFFSNVTWFHAIHV

SEQ ID NO: 25 - protein, 15 aa, Betacoronavirus severe acute respiratory syndrome coronavirus 2
SGTNGTKRFDNPVLP

SEQ ID NO: 26 - protein, 15 aa, Betacoronavirus severe acute respiratory syndrome coronavirus 2
FHAISGTNGTKRFDN

SEQ ID NO: 27 - protein, 15 aa, Betacoronavirus severe acute respiratory syndrome coronavirus 2
NVTWFHAISGTNGTK

SEQ ID NO: 28 - protein, 15 aa, Betacoronavirus severe acute respiratory syndrome coronavirus 2
PFFSNVTWFHAISGT

SEQ ID NO: 29 - protein, 15 aa, Betacoronavirus severe acute respiratory syndrome coronavirus 2
ISGTNGTKRFDNPVL

SEQ ID NO: 30 - protein, 15 aa, Betacoronavirus severe acute respiratory syndrome coronavirus 2
WFHAISGTNGTKRFD

SEQ ID NO: 31 - protein, 15 aa, Betacoronavirus severe acute respiratory syndrome coronavirus 2
SNVTWFHAISGTNGT

SEQ ID NO: 32 - protein, 15 aa, Betacoronavirus severe acute respiratory syndrome coronavirus 2
LPFFSNVTWFHAISG

SEQ ID NO: 33 - protein, 15 aa, Betacoronavirus severe acute respiratory syndrome coronavirus 2
NSPRRARSVASQSII

SEQ ID NO: 34 - protein, 15 aa, Betacoronavirus severe acute respiratory syndrome coronavirus 2
QTQTNSPRRARSVAS

SEQ ID NO: 35 - protein, 15 aa, Betacoronavirus severe acute respiratory syndrome coronavirus 2
CASYQTQTNSPRRAR

SEQ ID NO: 36 - protein, 15 aa, Betacoronavirus severe acute respiratory syndrome coronavirus 2
GAGICASYQTQTNSP

SEQ ID NO: 37 - protein, 15 aa, Betacoronavirus severe acute respiratory syndrome coronavirus 2
PRRARSVASQSIIAY

SEQ ID NO: 38 - protein, 15 aa, Betacoronavirus severe acute respiratory syndrome coronavirus 2
QTNSPRRARSVASQS

SEQ ID NO: 39 - protein, 15 aa, Betacoronavirus severe acute respiratory syndrome coronavirus 2
SYQTQTNSPRRARSV

SEQ ID NO: 40 - protein, 15 aa, Betacoronavirus severe acute respiratory syndrome coronavirus 2
GICASYQTQTNSPRR

SEQ ID NO: 41 - protein, 15 aa, Betacoronavirus severe acute respiratory syndrome coronavirus 2
NSHRRARSVASQSII

SEQ ID NO: 42 - protein, 15 aa, Betacoronavirus severe acute respiratory syndrome coronavirus 2
QTQTNSHRRARSVAS

SEQ ID NO: 43 - protein, 15 aa, Betacoronavirus severe acute respiratory syndrome coronavirus 2
CASYQTQTNSHRRAR

SEQ ID NO: 44 - protein, 15 aa, Betacoronavirus severe acute respiratory syndrome coronavirus 2
GAGICASYQTQTNSH

SEQ ID NO: 45 - protein, 15 aa, Betacoronavirus severe acute respiratory syndrome coronavirus 2
HRRARSVASQSIIAY

SEQ ID NO: 46 - protein, 15 aa, Betacoronavirus severe acute respiratory syndrome coronavirus 2
QTNSHRRARSVASQS

SEQ ID NO: 47 - protein, 15 aa, Betacoronavirus severe acute respiratory syndrome coronavirus 2
SYQTQTNSHRRARSV

SEQ ID NO: 48 - protein, 15 aa, Betacoronavirus severe acute respiratory syndrome coronavirus 2
GICASYQTQTNSHRR

SEQ ID NO: 49 - protein, 15 aa, Betacoronavirus severe acute respiratory syndrome coronavirus 2
PTNGVGYQPYRVVVL

SEQ ID NO: 50 - protein, 15 aa, Betacoronavirus severe acute respiratory syndrome coronavirus 2
YGFQPTNGVGYQPYR

SEQ ID NO: 51 - protein, 15 aa, Betacoronavirus severe acute respiratory syndrome coronavirus 2
PLQSYGFQPTNGVGY

SEQ ID NO: 52 - protein, 15 aa, Betacoronavirus severe acute respiratory syndrome coronavirus 2
NCYFPLQSYGFQPTN

SEQ ID NO: 53 - protein, 15 aa, Betacoronavirus severe acute respiratory syndrome coronavirus 2
NGVGYQPYRVVVLSF

SEQ ID NO: 54 - protein, 15 aa, Betacoronavirus severe acute respiratory syndrome coronavirus 2
FQPTNGVGYQPYRVV

SEQ ID NO: 55 - protein, 15 aa, Betacoronavirus severe acute respiratory syndrome coronavirus 2
QSYGFQPTNGVGYQP

SEQ ID NO: 56 - protein, 15 aa, Betacoronavirus severe acute respiratory syndrome coronavirus 2
YFPLQSYGFQPTNGV

SEQ ID NO: 57 - protein, 15 aa, Betacoronavirus severe acute respiratory syndrome coronavirus 2
PTYGVGYQPYRVVVL

SEQ ID NO: 58 - protein, 15 aa, Betacoronavirus severe acute respiratory syndrome coronavirus 2
YGFQPTYGVGYQPYR

SEQ ID NO: 59 - protein, 15 aa, Betacoronavirus severe acute respiratory syndrome coronavirus 2
PLQSYGFQPTYGVGY

SEQ ID NO: 60 - protein, 15 aa, Betacoronavirus severe acute respiratory syndrome coronavirus 2
NCYFPLQSYGFQPTY

SEQ ID NO: 61 - protein, 15 aa, Betacoronavirus severe acute respiratory syndrome coronavirus 2
YGVGYQPYRVVVLSF

SEQ ID NO: 62 - protein, 15 aa, Betacoronavirus severe acute respiratory syndrome coronavirus 2
FQPTYGVGYQPYRVV

SEQ ID NO: 63 - protein, 15 aa, Betacoronavirus severe acute respiratory syndrome coronavirus 2
QSYGFQPTYGVGYQP

SEQ ID NO: 64 - protein, 15 aa, Betacoronavirus severe acute respiratory syndrome coronavirus 2
YFPLQSYGFQPTYGV

SEQ ID NO: 65 - protein, 15 aa, Betacoronavirus severe acute respiratory syndrome coronavirus 2
GVEGFNCYFPLQSYG

SEQ ID NO: 66 - protein, 15 aa, Betacoronavirus severe acute respiratory syndrome coronavirus 2
TPCNGVEGFNCYFPL

SEQ ID NO: 67 - protein, 15 aa, Betacoronavirus severe acute respiratory syndrome coronavirus 2
QAGSTPCNGVEGFNC

SEQ ID NO: 68 - protein, 15 aa, Betacoronavirus severe acute respiratory syndrome coronavirus 2
TEIYQAGSTPCNGVE

SEQ ID NO: 69 - protein, 15 aa, Betacoronavirus severe acute respiratory syndrome coronavirus 2
EGFNCYFPLQSYGFQ

SEQ ID NO: 70 - protein, 15 aa, Betacoronavirus severe acute respiratory syndrome coronavirus 2
CNGVEGFNCYFPLQS

SEQ ID NO: 71 - protein, 15 aa, Betacoronavirus severe acute respiratory syndrome coronavirus 2
GSTPCNGVEGFNCYF

SEQ ID NO: 72 - protein, 15 aa, Betacoronavirus severe acute respiratory syndrome coronavirus 2
IYQAGSTPCNGVEGF

SEQ ID NO: 73 - protein, 15 aa, Betacoronavirus severe acute respiratory syndrome coronavirus 2
GVKGFNCYFPLQSYG

SEQ ID NO: 74 - protein, 15 aa, Betacoronavirus severe acute respiratory syndrome coronavirus 2
TPCNGVKGFNCYFPL

SEQ ID NO: 75 - protein, 15 aa, Betacoronavirus severe acute respiratory syndrome coronavirus 2
QAGSTPCNGVKGFNC

SEQ ID NO: 76 - protein, 15 aa, Betacoronavirus severe acute respiratory syndrome coronavirus 2
TEIYQAGSTPCNGVK

SEQ ID NO: 77 - protein, 15 aa, Betacoronavirus severe acute respiratory syndrome coronavirus 2
KGFNCYFPLQSYGFQ

SEQ ID NO: 78 - protein, 15 aa, Betacoronavirus severe acute respiratory syndrome coronavirus 2
CNGVKGFNCYFPLQS

SEQ ID NO: 79 - protein, 15 aa, Betacoronavirus severe acute respiratory syndrome coronavirus 2
GSTPCNGVKGFNCYF

SEQ ID NO: 80 - protein, 15 aa, Betacoronavirus severe acute respiratory syndrome coronavirus 2
IYQAGSTPCNGVKGF

SEQ ID NO: 81 - protein, 15 aa, Betacoronavirus severe acute respiratory syndrome coronavirus 2
TGKIADYNYKLPDDF

SEQ ID NO: 82 - protein, 15 aa, Betacoronavirus severe acute respiratory syndrome coronavirus 2
APGQTGKIADYNYKL

SEQ ID NO: 83 - protein, 15 aa, Betacoronavirus severe acute respiratory syndrome coronavirus 2
VRQIAPGQTGKIADY

SEQ ID NO: 84 - protein, 15 aa, Betacoronavirus severe acute respiratory syndrome coronavirus 2
RGDEVRQIAPGQTGK

SEQ ID NO: 85 - protein, 15 aa, Betacoronavirus severe acute respiratory syndrome coronavirus 2
KIADYNYKLPDDFTG

SEQ ID NO: 86 - protein, 15 aa, Betacoronavirus severe acute respiratory syndrome coronavirus 2
GQTGKIADYNYKLPD

SEQ ID NO: 87 - protein, 15 aa, Betacoronavirus severe acute respiratory syndrome coronavirus 2
QIAPGQTGKIADYNY

SEQ ID NO: 88 - protein, 15 aa, Betacoronavirus severe acute respiratory syndrome coronavirus 2
DEVRQIAPGQTGKIA

SEQ ID NO: 89 - protein, 15 aa, Betacoronavirus severe acute respiratory syndrome coronavirus 2
TGNIADYNYKLPDDF

SEQ ID NO: 90 - protein, 15 aa, Betacoronavirus severe acute respiratory syndrome coronavirus 2
APGQTGNIADYNYKL

SEQ ID NO: 91 - protein, 15 aa, Betacoronavirus severe acute respiratory syndrome coronavirus 2
VRQIAPGQTGNIADY

SEQ ID NO: 92 - protein, 15 aa, Betacoronavirus severe acute respiratory syndrome coronavirus 2
RGDEVRQIAPGQTGN

SEQ ID NO: 93 - protein, 15 aa, Betacoronavirus severe acute respiratory syndrome coronavirus 2
NIADYNYKLPDDFTG

SEQ ID NO: 94 - protein, 15 aa, Betacoronavirus severe acute respiratory syndrome coronavirus 2
GQTGNIADYNYKLPD

SEQ ID NO: 95 - protein, 15 aa, Betacoronavirus severe acute respiratory syndrome coronavirus 2
QIAPGQTGNIADYNY

SEQ ID NO: 96 - protein, 15 aa, Betacoronavirus severe acute respiratory syndrome coronavirus 2
DEVRQIAPGQTGNIA

SEQ ID NO: 97 - protein, 1273 aa, Betacoronavirus severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 98 - protein, 50 aa, Betacoronavirus severe acute respiratory syndrome coronavirus 2
GTNTSNQVAVLYQDVNCTEVPVAIHADQLTPTWRVYSTGSNVFQTRAGCL

SEQ ID NO: 99 - protein, 50 aa, Betacoronavirus severe acute respiratory syndrome coronavirus 2
GTNTSNQVAVLYQGVNCTEVPVAIHADQLTPTWRVYSTGSNVFQTRAGCL

SEQ ID NO: 100 - protein, 50 aa, Betacoronavirus severe acute respiratory syndrome coronavirus 2
TQDLFLPFFSNVTWFHAIHVSGTNGTKRFDNPVLPFNDGVYFASTEKSNI

SEQ ID NO: 101 - protein, 48 aa, Betacoronavirus severe acute respiratory syndrome coronavirus 2
TQDLFLPFFSNVTWFHAISGTNGTKRFDNPVLPFNDGVYFASTEKSNI

SEQ ID NO: 102 - protein, 50 aa, Betacoronavirus severe acute respiratory syndrome coronavirus 2
IGAEHVNNSYECDIPIGAGICASYQTQTNSPRRARSVASQSIIAYTMSLG

SEQ ID NO: 103 - protein, 50 aa, Betacoronavirus severe acute respiratory syndrome coronavirus 2
IGAEHVNNSYECDIPIGAGICASYQTQTNSHRRARSVASQSIIAYTMSLG

SEQ ID NO: 104 - protein, 50 aa, Betacoronavirus severe acute respiratory syndrome coronavirus 2
NGVGYQPYRVVVLSFELLHAPATVCGPKKSTNLVKNKCVNFNFNGLTGTG

SEQ ID NO: 105 - protein, 50 aa, Betacoronavirus severe acute respiratory syndrome coronavirus 2
YGVGYQPYRVVVLSFELLHAPATVCGPKKSTNLVKNKCVNFNFNGLTGTG

SEQ ID NO: 106 - protein, 50 aa, Betacoronavirus severe acute respiratory syndrome coronavirus 2
YLYRLFRKSNLKPFERDISTEIYQAGSTPCNGVEGFNCYFPLQSYGFQPT

SEQ ID NO: 107 - protein, 50 aa, Betacoronavirus severe acute respiratory syndrome coronavirus 2
YLYRLFRKSNLKPFERDISTEIYQAGSTPCNGVKGFNCYFPLQSYGFQPT

SEQ ID NO: 108 - protein, 50 aa, Betacoronavirus severe acute respiratory syndrome coronavirus 2
VIRGDEVRQIAPGQTGKIADYNYKLPDDFTGCVIAWNSNNLDSKVGGNYN

SEQ ID NO: 109 - protein, 50 aa, Betacoronavirus severe acute respiratory syndrome coronavirus 2
VIRGDEVRQIAPGQTGNIADYNYKLPDDFTGCVIAWNSNNLDSKVGGNYN